SEQUENCE LISTING (33 sequences)

SEQ ID NO: 1 - DNA, 20 bp, synthetic construct
  source 1..20: mol_type = other DNA
ccccagctcagcagataaca

SEQ ID NO: 2 - DNA, 20 bp, synthetic construct
  source 1..20: mol_type = other DNA
aggcttctcaggtgattgcg

SEQ ID NO: 3 - DNA, 20 bp, synthetic construct
  source 1..20: mol_type = other DNA
aagggcacagggagatgtca

SEQ ID NO: 4 - DNA, 20 bp, synthetic construct
  source 1..20: mol_type = other DNA
ggtgttgctcaaccttcggt

SEQ ID NO: 5 - DNA, 21 bp, synthetic construct
  source 1..21: mol_type = other DNA
gacaacgctggcacttttgta

SEQ ID NO: 6 - DNA, 20 bp, synthetic construct
  source 1..20: mol_type = other DNA
tgtgcaagcaggtcattgtg

SEQ ID NO: 7 - DNA, 21 bp, synthetic construct
  source 1..21: mol_type = other DNA
atcacttctcactaccggcat

SEQ ID NO: 8 - DNA, 23 bp, synthetic construct
  source 1..23: mol_type = other DNA
aagacgttcttatgtgccttgaa

SEQ ID NO: 9 - DNA, 20 bp, synthetic construct
  source 1..20: mol_type = other DNA
cgccttataaacctcccgct

SEQ ID NO: 10 - DNA, 20 bp, synthetic construct
  source 1..20: mol_type = other DNA
tggccacttccatgggtcta

SEQ ID NO: 11 - DNA, 23 bp, synthetic construct
  source 1..23: mol_type = other DNA
gatcgtgtttttcagagtctcca

SEQ ID NO: 12 - DNA, 22 bp, synthetic construct
  source 1..22: mol_type = other DNA
tgcagtcaaccccttgaataag

SEQ ID NO: 13 - DNA, 21 bp, synthetic construct
  source 1..21: mol_type = other DNA
caggttcaacgaccttcgatt

SEQ ID NO: 14 - DNA, 20 bp, synthetic construct
  source 1..20: mol_type = other DNA
gtggtaggtagccacttggg

SEQ ID NO: 15 - DNA, 20 bp, synthetic construct
  source 1..20: mol_type = other DNA
tccgacagtggttgatcgac

SEQ ID NO: 16 - DNA, 22 bp, synthetic construct
  source 1..22: mol_type = other DNA
cctcacgattgtagtcctgctt

SEQ ID NO: 17 - DNA, 22 bp, synthetic construct
  source 1..22: mol_type = other DNA
aactgactgctcgcaataatgt

SEQ ID NO: 18 - DNA, 22 bp, synthetic construct
  source 1..22: mol_type = other DNA
gtaacacagcaatgcctcttgt

SEQ ID NO: 19 - DNA, 22 bp, synthetic construct
  source 1..22: mol_type = other DNA
ctggatagcctttcttctgctg

SEQ ID NO: 20 - DNA, 20 bp, synthetic construct
  source 1..20: mol_type = other DNA
gcacactgtgtccgaactca

SEQ ID NO: 21 - DNA, 20 bp, synthetic construct
  source 1..20: mol_type = other DNA
tgtacgagtcggtgtgcttc

SEQ ID NO: 22 - DNA, 23 bp, synthetic construct
  source 1..23: mol_type = other DNA
ggtaggtatccgtcatggtcttg

SEQ ID NO: 23 - DNA, 21 bp, synthetic construct
  source 1..21: mol_type = other DNA
tacgtcggcagactcgatatt

SEQ ID NO: 24 - DNA, 21 bp, synthetic construct
  source 1..21: mol_type = other DNA
tgggcgtatctctctctcctt

SEQ ID NO: 25 - DNA, 20 bp, synthetic construct
  source 1..20: mol_type = other DNA
cagggcctttaccagggaga

SEQ ID NO: 26 - DNA, 21 bp, synthetic construct
  source 1..21: mol_type = other DNA
tgtgtgctggatgtgatcttc

SEQ ID NO: 27 - DNA, 22 bp, synthetic construct
  source 1..22: mol_type = other DNA
ttcgaccttctcaagttcgacg

SEQ ID NO: 28 - DNA, 21 bp, synthetic construct
  source 1..21: mol_type = other DNA
tcgagatgggtcttcggttca

SEQ ID NO: 29 - DNA, 19 bp, synthetic construct
  source 1..19: mol_type = other DNA
ctgccgtccgattgagacc

SEQ ID NO: 30 - DNA, 20 bp, synthetic construct
  source 1..20: mol_type = other DNA
cccctccttgtaccactgtc

SEQ ID NO: 31 - DNA, 22 bp, synthetic construct
  source 1..22: mol_type = other DNA
ctgatcttcttgctactcccca

SEQ ID NO: 32 - DNA, 21 bp, synthetic construct
  source 1..21: mol_type = other DNA
ggtgtagttcaagtgaggcag

SEQ ID NO: 33 - DNA, 1305 bp, Mus musculus
  source 1..1305: mol_type = genomic DNA
atgaacaaccgaaaggaagatatggaaatcacttctcactaccggcatctgcttcgagagctcaatgagcagaggcagcacggagtcctctgtgatgcgtgcgtcgtggtggagggcaaggtcttcaaggcacataagaacgtcttgcttgggagcagccgctactttaagacgctctactgccaggtacagaagacatctgaccaggccaccgtcactcacttggacattgttacagcccagggcttcaaggccattattgacttcatgtactccgcccatctggctctcactagtaggaatgtcatcgaggtgatgtcagctgccagcttcctacagatgactgacattgtgcaggcctgccatgatttcatcaaggctgcactggacatcagcataaagtcagatgcctccgatgaactctcagaatttgagattggcaccccagccagcaacagtacagaggcgttgatctcagctgtgatggctggaaggagtatctccccatggttggctcggagaacaagtcctgccaattcttctggagactctgccattgccagctgtcatgaaggaggaagcagctatgggaaggaggaccaggaacccaaagctgatggccctgatgacgtttcttcacagtctttgtggcctggagatgtaggctatgggtctctgcgcatcaaggaagaacagatttcaccatcacattatggagggagtgagcttccatcttccaaggacactgcaatacagaattctttatcagaacagggttctggggatggctggcagcccacaggccggaggaagaatcggaaaaacaaagagactgtccgacacatcacccagcaggtggaggaggacagccaggctggctctccagtaccttcattcctacccacatcgggatggcctttcagcagccgagactcaaatgtagacctgacggtcactgaggccagcagcttggacagccgaggcgagagagcagagctctatgctcacatcgatgagggcctactaggaggagaaaccagctacttgggcccacccctcaccccagagaaggaagaagcactacaccaggctactgcagtggccaatcttcgtgctgcactcatgagtaagaacagtctgctgtcactcaaggctgacgtgctcggtgatgatggctcacttctgttcgagtacctgcccaaaggtgcccactcactgtctcgtaagtgcaagttctggtgtgtcactgtgtcttcctttggtttaagcacctcagttcagcccttcagaccctggagtcactga

(19) United States
(12) Patent Application Publication
(10) Pub. No.: US 2024/0325493 A1
(43) Pub. Date: Oct. 3, 2024

(54) REGULATOR OF TUMOR CELL FUNCTIONS AND USES THEREOF

(71) Applicant: Washington University, St. Louis, MO (US)

(72) Inventors: Kyunghee Choi, St. Louis, MO (US); Karen Krchma, St. Louis, MO (US); Ashraf Ul Kabir, St. Louis, MO (US)

(73) Assignee: Washington University, St. Louis, MO (US)

(21) Appl. No.: 18/623,995

(22) Filed: Apr. 1, 2024

Related U.S. Application Data
(60) Provisional application No. 63/493,156, filed on Mar. 30, 2023.

(51) Int. Cl. A61K 38/18; A61K 39/395; A61K 48/00; A61P 35/00; C12Q 1/6886

(52) U.S. Cl. CPC A61K 38/18; A61K 39/39558; A61P 35/00; C12Q 1/6886; A61K 48/00; C12Q 2600/106; C12Q 2600/158

(57) ABSTRACT

Among the various aspects of the present disclosure is the provision of a regulator of tumor cell functions and uses thereof. Methods to treat a cancer patient with immunotherapy and a Zbtb46 modulation agent are disclosed. A method to identify a cancer patient in need of an adjuvant therapy to an immunotherapy is also disclosed.

### CROSS-REFERENCE TO RELATED APPLICATIONS

This application claims the benefit of priority to U.S. Provisional Application Ser. No. 63/493,156 filed on Mar. 30, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

### FIELD OF THE INVENTION

The present disclosure generally relates to methods of identifying cancer patients in need of adjuvant therapy with immunotherapy and methods of treating cancer patients with adjuvant therapy with immunotherapy.

### BACKGROUND OF THE INVENTION

The cooperation between tumor cells and the tumor microenvironment (TME) is a defining characteristic of cancer. As such, targeting the communication between the tumor and TME has recently gained much attention. One such strategy is immune checkpoint blockade (ICB) immunotherapy, which has significantly impacted the cancer treatment landscape by promoting long-term tumor control and complete regression in some patients. Unfortunately, most patients still do not respond to ICB therapies. The efficacy of ICB relies on the ability of T cells to kill cancer cells and the support of the vasculature and antigen-presenting cells, such as dendritic cells (DCs), in delivering and activating these T cells, respectively. Tumors can disrupt this process by suppressing anti-tumor programs in the TME components. Identifying these suppressive programs has tremendous potential for use as adjuvants to improve the outcomes of immunotherapies.

Zbtb46 is a member of the BTB-ZF family of transcriptional repressors and is considered a marker of classical DCs. Endothelial cells (ECs) of the vascular system also constitutively express Zbtb46. In homeostatic conditions, Zbtb46 is thought to keep both these cell types in a quiescent state. However, its function has not been addressed in any pathological conditions.

### SUMMARY OF THE INVENTION

Among the various aspects of the present disclosure is the provision of a regulator of tumor cell functions and uses thereof.

Briefly, therefore, the present disclosure is directed to methods of treatment for a cancer patient and methods of identifying cancer patients in need of adjuvant therapy to immunotherapy.

In one aspect, a method to treat a cancer in a patient is disclosed that includes administering a therapeutically effective amount of an immunotherapy and a therapeutically effective amount of at least one Zbtb46 modulation agent to the patient. In some aspects, the immunotherapy comprises an immune checkpoint blockade (ICB) therapy. In some aspects, the ICB therapy comprises an anti-PD1 therapy. In some aspects, the Zbtb46 modulation agent comprises a Zbtb46 overexpression construct, wherein the Zbtb46 overexpression construct increases the expression of Zbtb46 in a cancer patient's tumor microenvironment. In some aspects, the Zbtb46 modulation agent normalizes tumor blood vessels. In some aspects, the Zbtb46 modulation agent comprises a Cebpb antibody, wherein the Cebpb antibody suppresses Cebpb transcription. In some aspects, the Zbtb46 modulating agent comprises any one of BMS493, NS398, NAC+APO, or DC101, wherein the Zbtb46 modulating agent suppresses tumor-derived factors. In some aspects, the Zbtb46 modulation agent comprises a Cebpb shRNA construct or Cebpb antibody, wherein the Zbtb46 modulation agent enhances the generation of dendritic cells relative to macrophages from bone-marrow precursors. In some aspects, the Zbtb46 modulation agent enhances an immune response in a cancer patient's tumor. In some aspects, the Zbtb46 modulation agent comprises a Zbtb46 mRNA. In some aspects, the Zbtb46 modulation agent comprises a Zbtb46 mRNA nanoparticle.

In another aspect, a method of selecting a treatment for a cancer patient in need is disclosed that includes performing RNAseq on a biopsy of a tumor of the patient; quantifying an expression of Zbtb46 in the patient's tumor; and selecting a treatment of an immunotherapy and an adjuvant therapy for the patient if the expression of Zbtb46 falls below a standard threshold. In some aspects, the adjuvant therapy comprises a Zbtb46 modulation agent. In some aspects, The method of claim 12, wherein the Zbtb46 modulation agent comprises a Zbtb46 mRNA. In some aspects, the immunotherapy comprises an ICB therapy. In some aspects, the ICB therapy comprises an anti-PD1 therapy. In some aspects, the method further includes identifying the standard threshold by comparing a Zbtb46 expression in patients that respond to the immunotherapy to a corresponding Zbtb46 expression in patients that do not respond to immunotherapy, and selecting the threshold expression value that most differentiates the patients that do not respond to the immunotherapy from the patients that do respond to the immunotherapy.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a set of graphs of Zbtb46 mRNA expression in (left) CD31+CD45−ECs, (middle) CD45+CD11c+MHCII+DCs, and (right) bone marrow cells. Cells were isolated from the lungs (LEC), Spleen (SDC), and bone (H. BM) of the healthy wild-type mice or from the tumors (TEC and TDC) and bone of tumor bearing mice (T. BM) of the 1956 sarcoma-bearing wild-type mice at indicated days post-transplantation. n≥3/group. Data are mean±SD. (left and middle) One-way ANOVA with Dunnett's test and (right) Student's t-test.

FIG. 1B is a graph of tumor growth in WT and Zbtb46 KO mice with 1956 sarcomas. n≥8/group. Student's t-test at endpoint.

FIG. 1C is a graph of tumor growth in WT and Zbtb46 KO mice with PyMT-BO1 tumors. n≥8/group. Student's t-test at endpoint.

FIG. 1D is a graph of tumor growth in WT and Zbtb46 KO mice with LLC carcinomas. n≥8/group. Student's t-test at endpoint.

FIG. 1E is a graph of tumor growth in WT and Zbtb46 KO mice with MMTV-PyMT tumors. n≥8/group. Student's t-test at endpoint.

FIG. 1F contains representative images and accompanying graphs quantifying tumoral CD31+ vessel density in wild-type and Zbtb46 KO mice bearing 1956 sarcoma (top, left), PyMT-BO1 (top, right), LLC carcinoma (bottom, left), and MMTV-PyMT (bottom, right). n≥4/group. Data are mean±SD. Student's t-test.

FIG. 1G contains graphs measuring the ratio of CTL/Treg, the ratio of M1/M2 macrophages, cDC1 cells (CD11c+MHCII+XCR1+), and Fibroblast activation protein (FAP)+ in the TME of 1956 sarcoma (top) and PyMT-BO1 (bottom) breast tumor-bearing mice at endpoint (day 21). n=4/group. Data are mean±SD. Student's t-test.

FIG. 1H is a set of graphs of CD8 T cells (top) and classical dendritic cells (bottom) in high (red) vs. low (blue) ZBTB46 expressing tumors in patients from the TCGA database analyzed with XCell. Data are mean±SEM. Student's t-test. *p<0.05, **p<0.01, ***p<0.001, ns=not significant.

FIG. 1I is a set of graphs of 1956 sarcoma (left) and PyMT-Bo1 (right) tumor growth in WT and Zbtb46 KO mice with a bone marrow transplant from a WT or Zbtb46 KO donor. n≥5/group. One-way ANOVA with Dunnett's test at endpoint compared to control mice.

FIG. 1J is a set of graphs of 1956 sarcoma (left) and PyMT-Bo1 (right) tumor growth in WT, VEC-cre Zbtb46 KO, CD11c-cre Zbtb46 KO, and Zbtb46 KO mice. One-way ANOVA with Dunnett's test at endpoint compared to wild-type mice.

FIG. 1K is a schematic and timeline depicting tumor inoculation followed by Zbtb46 lentivirus injection into the tumor.

FIG. 1L is a graph of tumor growth of 1956 sarcoma in wild-type mice with either empty vector (EV) or Zbtb46 (ZOE) lentiviral overexpression construct (intra-tumor) treatment. n≥8/group. Student's t-test at endpoint compared to EV-treated mice.

FIG. 1M is a graph of tumor growth of PyMT-BO1 in wild-type mice with either empty vector (EV) or Zbtb46 (ZOE) lentiviral overexpression construct (intra-tumor) treatment. n≥8/group. Student's t-test at endpoint compared to EV-treated mice.

FIG. 1N is a graph of tumor growth of LLC carcinoma in wild-type mice with either empty vector (EV) or Zbtb46 (ZOE) lentiviral overexpression construct (intra-tumor) treatment. n≥8/group. Student's t-test at endpoint compared to EV-treated mice.

FIG. 2A is a set of representative images with graphs quantifying tumoral CD31+ vessel density in wild-type mice with either empty vector (WT+EV) or Zbtb46 (WT+ZOE) lentiviral overexpression construct (intra-tumor) treatment bearing 1956 sarcoma (top), LLC carcinoma (middle), and PyMT-BO1 (bottom) tumors. n≥4/group. Data are mean±SD. Student's t-test.

FIG. 2B is a set of representative images with graphs quantifying vascular leakage (top) and intra-tumoral hypoxia (bottom) as measured by the FITC-Dextran 70KD spread and the relative abundance of Hypoxyprobe-1 binding, respectively, in the 1956 sarcoma tumor tissue of wild-type mice with either empty vector (WT+EV) or Zbtb46 (WT+ZOE) or of Zbtb46 KO mice with empty vector (ZKO+EV) lentiviral overexpression construct (intra-tumor) treatment. n≥4/group. Data are mean±SD. One-way ANOVA with Dunnett's test.

FIG. 2C is a set of graphs of GO enrichment analysis of bulk-RNA sequencing data from parental (WT) (left) and Zbtb46-overexpressed (OE) (right) MCEC (mouse cardiac endothelial cells).

FIG. 2D is a schematic (left) and associated graph (right) of the relative migration of CD3+CD8+ CTL cells through parental (WT) and Zbtb46 overexpressed (ZOE) MCEC cell barrier in a leukocyte trans-endothelial migration assay. Data are mean±SD. Student's t-test.

FIG. 2E is a schematic (left) and graph (right) of tumor growth of 1956 sarcoma cells co-transplanted with either parental ECs (TC+WTEC) or Zbtb46-overexpressed ECs (TC+ZOEEC). N≥6/group. Student's t-test at endpoint.

FIG. 3A is a graph of tumor growth of 1969 regressive sarcoma in wild-type, VEC-cre Zbtb46 KO, VAV-cre Zbtb46 KO, and Zbtb46 KO mice. N≥6/group.

FIG. 3B is a set of graphs depicting the percent of GMP and Pre-cDC cells from bone marrow (BM) (left), the percent of GMP, Pre-cDC, and cDC cells from spleen (middle), and the percent of CD3, cDC, and Gr1+ cells from peripheral blood (PB) (right) in WT and Zbtb46 KO (ZKO) mice bearing 1956 sarcoma. GMP: Lin-Sca1-cKit+CD41-CD150-CD16/32+, Pre-cDC: Lin-Sca1-IL7R-MHCII-CD16/32-CD11c+cKit-CD135+, cDC: CD11c+MHCII+. N≥3/group. Data are mean±SD. Student's t-test.

FIG. 3C is a schematic (top) and sets of graphs (middle and bottom). The schematic depicts a timeline of tumor inoculation followed by Zbtb46 lentivirus injection into the tumor and then sacrifice and analysis. The graphs show the CTL/Treg ratio, M1/M2 macrophage ratio, percent of cDC1 cells, percent of NK cells, and percent of FPA+ cells in 1956 sarcoma-bearing (middle) and PyMT-BO1 tumor-bearing (bottom) wild-type mice with either empty vector (EV) or Zbtb46 (ZOE) lentiviral overexpression construct (intra-tumor) treatment. n≥4/group. Data are mean±SD. Student's t-test.

FIG. 3D is a schematic and associated set of graphs showing the analysis of Gr1+ (left graph) and MHCII+ (right graph) cell generation from wild-type or Zbtb46 KO mice bone marrow cells with either empty vector-mCherry (WT and ZKO) or Zbtb46-mCherry (ZOE) lentiviral overexpression. n=3/group. Data are mean±SD. One-way ANOVA with Dunnett's test.

FIG. 3E is a flowchart and associated set of graphs showing the analysis of Gr1+ (left graph) and MHCII+ (right graph) cell generation from wild-type or Zbtb46 KO mice KSL cells (Lin-Sca1+cKit+) sorted from bone marrow. n=4/group. Data are mean±SD. Student's t-test.

FIG. 3F is a schematic and associated set of graphs showing the analysis of donor-derived tumor-associated macrophages (TAM) and DCs after five days of intra-tumor transfer of enriched monocytes from wild-type donor mice bone marrow (CD45.1) into 1956 sarcoma-bearing wild-type recipient mice (CD45.2) with either empty vector-mCherry (WT+EV) or Zbtb46-mCherry (WT+ZOE) lentiviral overexpression. n≥5/group. Data are mean±SD. Student's t-test.

FIG. 3G is a graph of ChIP-qPCR analysis of ZBTB46 recruitment to a potential binding site in the Cebpb promoter region. Zbtb46-HA lentiviral construct was overexpressed in wild-type mice bone marrow-derived precursors (monocyte and lineage-negative cells) and ChIP was performed with an anti-HA antibody. n≥3/group. Data are mean±SD. Student's t-test.

FIG. 3H is a set of graphs of Gr1+ and MHCII+ cell generation from bone marrow cells of Zbtb46 KO mice with empty vector-mCherry lentiviral overexpression alone (ZKO) or with Cebpb shRNA constructs (ZKO+CKD) and of wild-type mice with Zbtb46-mCherry lentiviral overexpression alone (ZOE) or with Cebpb overexpression (ZOE+COE). n=3/group. Data are mean±SD. Student's t-test.

FIG. 3I is a set of graphs of ChIP-qPCR analysis for CEBPB enrichment at CEBPB peaks in bone marrow-derived precursors (monocyte and lineage-negative cells) from wild-type or Zbtb46 KO mice. n≥3/group. Data are mean±SD. Student's t-test.

FIG. 3J is a graph of tumor progression after five days of intra-tumoral transfer of enriched monocytes from wild-type mice bone marrow into 1956 sarcoma-bearing wild-type mice with either empty vector (EV) or Zbtb46 (ZOE) lentiviral overexpression. n≥6/group. Data are mean±SD. Student's t-test.

FIG. 4A is a schematic of wild-type tumor-bearing mice treatment with Zbtb46 mRNA nanoparticle, anti-PD1, and DC101 (VEGFR2 inhibitor).

FIG. 4B is a set of graphs of tumor growth of 1956 sarcoma (left) and PyMT-BO1 (right) breast cancer in wild-type mice with Zbtb46 mRNA nanoparticle (ZmR), anti-PD1 (AP), and DC101 (DC) treatment at 12 days post-treatment initiation. n≥6/group. Data are mean±SD. One-way ANOVA with Dunnett's test.

FIG. 4C is a set of graphs of tumor growth kinetics in 1956 sarcoma in wild-type mice treated with, from left to right, vehicle control, Zbtb46 mRNA nanoparticle, anti-PD1, and DC101, DC101+Anti-PD1, ZmR+Anti-PD1, and ZmR+DC101+Anti-PD1. CR=complete remission.

FIG. 4D is a set of graphs of tumor growth kinetics in PyMT-BO1 in wild-type mice treated with, from left to right, vehicle control, Zbtb46 mRNA nanoparticle, anti-PD1, and DC101, DC101+Anti-PD1, ZmR+Anti-PD1, and ZmR+DC101+Anti-PD1. CR-complete remission.

FIG. 5A is a set of graphs of vital cardiovascular parameters measuring, from left to right, heart rate, systolic BP, Diastolic BP, Mean arterial BP, and Pulse pressure in the Zbtb46 KO and wild-type mice. n≥4/group. Data are mean±SD. Student's t-test.

FIG. 5B is a set of graphs of pressure-diameter measurements characterizing carotid artery compliance (left) and aortic artery compliance (right) in the Zbtb46 KO and wild-type mice. N≥4/group. Data are mean±SD. One-way ANOVA with Tukey's multiple comparison test.

FIG. 5C is a set of graphs of Zbtb46 mRNA expression in (left) CD31+CD45-ECs and (right) CD45+CD11c+MHCII+DCs isolated from the mammary gland (MEC) and Spleen (SDC) of the healthy wild-type mice and from the tumors (TEC and TDC) of the PyMT-BO1-bearing wild-type mice at indicated days post-transplantation. N≥3/group. Data are mean±SD. One-way ANOVA with Dunnett's test.

FIG. 5D is a graph of Zbtb46 mRNA expression in CD31+CD45-ECs isolated from the lungs (LEC) of the healthy wild-type mice and from the tumors (TEC) of the LLC carcinoma bearing mice. N≥3/group. Data are mean±SD. Student's t-test.

FIG. 5E is a graph of Zbtb46 mRNA expression in CD31+CD45-ECs isolated from and mammary gland (MEC) of the healthy wild-type mice and from the tumors (TEC) of the MMTV-PyMT bearing mice. N≥3/group. Data are mean±SD. Student's t-test.

FIG. 5F is a set of FACs plots (left) and associated graph (right) of ZBTB46+ (GFP+) CD31+CD45-ECs isolated from the lungs of healthy mice and tumors of the 1956-bearing Zbtb46gfp/+ mice. n≥3/group. Data are mean±SD. Student's t-test.

FIG. 5G is a set of FACs plots (left) and associated graph (right) of ZBTB46+ (GFP+) CD45+CD11c+MHCII+DCs isolated from the spleens of healthy mice and tumors of the 1956-bearing Zbtb46gfp/+ mice. n≥3/group. Data are mean±SD. Student's t-test.

FIG. 6A is a set of graphs of the percent of B, CD8T, Treg, and cDC1 cells in the tumor-draining lymph nodes of 1956 sarcoma-bearing mice. n=3/group. Data are mean±SD. Student's t-test.

FIG. 6B is a set of survival curves of the overall survival of patients with indicated cancers separated by ZBTB46 expression as high (>50th percentile) and low (<50th percentile). Survival data were derived from publicly available clinical records of TCGA patients. Log ranked test was used for survival analysis.

FIG. 6C is a set of graphs of the presence of, from top to bottom, activated dendritic cells, M1 macrophages, B cells, and CD8+T effector memory cells in high vs. low ZBTB46 expressing tumors in patients from the TCGA database analyzed with the CIBERSORT algorithm. Data are mean±SEM. Student's t-test. *p<0.05, **p<0.01, ***p<0.001, ns=not significant.

FIG. 7A contains schematics, associated flow cytometry plots (top), and summary graphs (bottom) of bone marrow chimera mice with graphs quantifying CD45.1 (for stromal KO) or CD45.2 (for hematopoietic KO) repopulation in the peripheral blood. n≥3/group. Data are mean±SD. One-way ANOVA with Dunnett's test.

FIG. 7B is a set of graphs of tumor growth of 1956 sarcoma (left) and PyMT-BO1 (right) breast cancer in wild-type, VEC-cre Zbtb46 KO, VAV-cre Zbtb46 KO, and Zbtb46 KO mice. n≥3/group. One-way ANOVA with Dunnett's test at endpoint compared to wild-type mice.

FIG. 7C is a set of graphs of Zbtb46 mRNA expression in CD31+CD45−ECs, CD45+CD11c+MHCII+DCs, and GFP+ tumor cells (TC) isolated from the tumors of the 1956 sarcoma (left) and PyMT-BO1 (right)-bearing wild-type mice with empty vector (EV) or Zbtb46 (ZOE) lentiviral overexpression construct intra-tumor treatment. n≥3/group. Data are mean±SD. Student's t-test.

FIG. 7D is a set of graphs of tumor growth of 1956 sarcoma (left) and PyMT-BO1 (right) with empty vector (1956-EV and BO1-EV) or Zbtb46 (1956-ZOE and BO1-ZOE) lentiviral overexpression or Zbtb46 shRNA construct expression (1956-ZKD and BO1-ZKD) in wild-type mice. n≥4/group. One-way ANOVA with Dunnett's test at the endpoint.

FIG. 8A is a set of representative images and an associated graph quantifying vascular perfusion as measured by the FITC-lectin binding to vessels in the 1956 sarcoma tumor tissue of wild-type mice with either empty vector (WT+EV) or Zbtb46 (WT+ZOE) or of Zbtb46 KO mice with empty vector (ZKO+EV) lentiviral overexpression construct (intra-tumor) treatment. n≥4/group. Data are mean±SD. One-way ANOVA with Dunnett's test.

FIG. 8B is a graph of proliferation by counting cell numbers of the cultured parental, Zbtb46 knockdown (ZKD), and Zbtb46 overexpressing (ZOE) MCEC cells. n≥3/group. Data are mean±SD. One-way ANOVA with Dunnett's test.

FIG. 8C is a set of representative images of a Matrigel tube formation assay with parental, Zbtb46 knockdown (ZKD), and Zbtb46 overexpressing (ZOE) MCEC cells.

FIG. 9A is a graph of tumor growth of 1969 regressive sarcoma in wild-type, VEC-cre Zbtb46 KO, CD11c-cre Zbtb46 KO, and Zbtb46 KO mice. n≥4/group.

FIG. 9B is a set of graphs of hemavet analysis and flow-cytometric analysis of peripheral blood (PB) (top), bone marrow (BM) (bottom left), and spleen (bottom right) in the wild-type and Zbtb46 KO mice in healthy condition. n≥3/group. Data are mean±SD. Student's t-test.

FIG. 10A is a genomic snapshot image depicting the ZBTB46 binding regions at the indicated genomic loci.

FIG. 10B is a graph of Cebpb mRNA expression in the bone marrow cells of wild-type or Zbtb46 KO mice with either empty vector (EV) or Zbtb46 (ZOE) lentiviral overexpression. n=3/group. Data are mean±SD. One-way ANOVA with Dunnett's test.

FIG. 10C is a set of graphs of Zbtb46 (left) and Cebpb (right) mRNA expression in the bone marrow cells of wild-type or Zbtb46 KO mice with either empty vector (EV), or Zbtb46 (ZOE), or Cebpb shRNA constructs (CKD), or Cebpb (COE) lentiviral overexpression. n=3/group. Data are mean±SD. One-way ANOVA with Dunnett's test.

FIG. 10D is an image of consensus motifs for ZBTB46 and CEBPB ChIP sequences.

FIG. 10E contains genomic snapshot images depicting the CEBPB binding regions at the CEBPB (top) and CSF3R (bottom) loci.

FIG. 10F is a schematic of the reporter lentivector core expression cassette for the CEBP signaling pathway.

FIG. 10G is a graph of chemiluminescence measurement of SEAP activity in the reporter-only (control), reporter with Cebpb overexpressed (COE), or reporter with Cebpb and Zbtb46 overexpressed (COE+ZOE) assay system. Data are mean±SD. One-way ANOVA with Dunnett's test.

FIG. 10H is a set of graphs of DC signature genes in 1956 sarcoma-bearing bone marrow cells from wild-type and Zbtb46 KO mice with empty vector (EV) or Zbtb46 (ZOE) overexpression. n=3/group. Data are mean±SD. One-way ANOVA with Dunnett's test.

FIG. 10I is a set of graphs of macrophage signature genes in 1956 sarcoma-bearing bone marrow cells from wild-type and Zbtb46 KO mice with empty vector (EV) or Zbtb46 (ZOE) overexpression. n=3/group. Data are mean±SD. One-way ANOVA with Dunnett's test.

FIG. 11A is a schematic of wild-type tumor-bearing mice treatment with Zbtb46 mRNA nanoparticle.

FIG. 11B is a graph of tumor growth kinetics of 1956 sarcoma in wild-type mice with Zbtb46 mRNA nanoparticle (ZmR) treatment. n≥3/group. Data are mean±SD. Student's t-test at endpoint.

FIG. 11C is a set of graphs of Zbtb46 mRNA expression in tumor endothelial cells (left), tumor dendritic cells (middle), and tumor cells (right) from wild-type mice bearing 1956 sarcoma treated with Zbtb46 mRNA nanoparticle (ZmR). n≥3/group. Data are mean±SD. Student's t-test at endpoint.

FIG. 11D is a set of graphs of immune microenvironment markers, in order from left to right, CTL/Treg ratio, M1/M2 ratio, cDC1 cells, and NK cells from wild-type mice bearing 1956 sarcoma treated with Zbtb46 mRNA nanoparticle (ZmR). n≥3/group. Data are mean±SD. Student's t-test at endpoint.

FIG. 11E is a graph of tumor growth kinetics of PyMT-BO1 breast cancer in wild-type mice with Zbtb46 mRNA nanoparticle (ZmR) treatment. n≥3/group. Data are mean±SD. Student's t-test at endpoint.

FIG. 11F is a set of graphs of Zbtb46 mRNA expression in tumor endothelial cells (left), tumor dendritic cells (middle), and tumor cells (right) from wild-type mice bearing PyMT-BO1 tumors treated with Zbtb46 mRNA nanoparticle (ZmR). n≥3/group. Data are mean±SD. Student's t-test at endpoint.

FIG. 11G is a set of graphs of immune microenvironment markers, in order from left to right, CTL/Treg ratio, M1/M2 ratio, cDC1 cells, and NK cells from wild-type mice bearing PyMT-BO1 tumors treated with Zbtb46 mRNA nanoparticle (ZmR). n≥3/group. Data are mean±SD. Student's t-test at endpoint.

FIG. 11H is a schematic and associated graph of tumor growth in the treatment-responded 1956 sarcoma tumor-eliminated mice challenged with secondary 1956 sarcoma transplantation.

FIG. 11I is a schematic and associated set of graphs of tumor growth of 1956 sarcoma cells co-transplanted with parental endothelial cells (WTEC) or Zbtb46 overexpressed endothelial cells (ZOEEC) with IgG or anti-PD1 treatment. n≥5/group. CR=Complete Remission. One-way ANOVA with Dunnett's test at 12 days post-treatment initiation.

FIG. 12A is a schematic of the TME and different distal organs from 1956 sarcoma-bearing wild-type mice with Zbtb46 mRNA nanoparticle (ZmR) and anti-PD1 treatment.

FIG. 12B is a set of graphs of hemavet and flow cytometric analysis of tumors for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 12C is a set of graphs of hemavet and flow cytometric analysis of tumor draining lymph node (TdLN) for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 12D is a set of graphs of hemavet and flow cytometric analysis of bone marrow (BM) for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 12E is a set of graphs of hemavet and flow cytometric analysis of the spleen for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 12F is a set of graphs of hemavet and flow cytometric analysis of lungs for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 12G is a set of graphs of hemavet and flow cytometric analysis of peripheral blood (PB) for immune components and precursor cells from 1956 sarcoma-bearing wild-type mice with Zbtb46 mRNA nanoparticle (ZmR) and anti-PD1 treatment. n≥3/group. One-way ANOVA with Dunnett's test.

FIG. 13A is a set of graphs of Zbtb46 mRNA expression in MCEC cells, with loading control b-Actin (left) and Gapdh (right), treated with PBS or tumor-conditioned media (TCM) for 24 hours. n≥3/group. One-way ANOVA with Dunnett's test.

FIG. 13B is a graph of Zbtb46 mRNA expression in bone marrow-derived dendritic cells (BMDC) treated with PBS, prostaglandin E2 (PGE2), vascular endothelial growth factor (VEGF), or retinoic acid (RA) for 24 hours. n≥3/group. One-way ANOVA with Dunnett's test.

FIG. 13C is a schematic depicting a treatment plan for tumor-bearing mice.

FIG. 13D is a set of graphs of tumor growth kinetics of 1956 sarcoma in wild-type mice treated with (left) BMS493 (inverse pan-retinoic acid receptor agonist), (middle) NS398 (selective cyclooxygenase-2 inhibitor), and (right) NAC+APO (ROS scavengers). n≥6/group. Data are mean±SD. Student's t-test at endpoint.

FIG. 13E is a set of graphs of Zbtb46 mRNA expression from tumor EC (left) and tumor DC (right) of 1956 sarcoma in wild-type mice treated with BMS493 (inverse pan-retinoic acid receptor agonist), NS398 (selective cyclooxygenase-2 inhibitor), and NAC+APO (ROS scavengers). n≥6/group. Data are mean±SD. Student's t-test at endpoint.

FIG. 13F is a set of graphs of tumor growth kinetics of PyMT-BO1 breast cancer in wild-type mice treated with (left) BMS493 (inverse pan-retinoic acid receptor agonist), (middle) NS398 (selective cyclooxygenase-2 inhibitor), and (right) NAC+APO (ROS scavengers). n≥6/group. Data are mean±SD. Student's t-test at endpoint.

FIG. 13G is a set of graphs of Zbtb46 mRNA expression from tumor EC (left) and tumor DC (right) of PyMT-BO1 breast cancer in wild-type mice treated with BMS493 (inverse pan-retinoic acid receptor agonist), NS398 (selective cyclooxygenase-2 inhibitor), and NAC+APO (ROS scavengers). n≥6/group. Data are mean±SD. Student's t-test at endpoint.

### DETAILED DESCRIPTION OF THE INVENTION

The present disclosure is based, at least in part, on the discovery that Zbtb46 downregulation leads to a stereotypical pro-tumor microenvironment. As shown herein, methods to identify cancer patients with pro-tumor microenvironments and methods of treating such patients with an adjuvant in combination with immunotherapy are disclosed.

One aspect of the present disclosure provides for a method of treatment for cancer that includes administering an immunotherapy, which can include but is not limited to an immune checkpoint blockade (ICB) such as anti-PD1 therapy, and a Zbtb46 modulation agent. Zbtb46 expression orchestrates a critical tumor-suppressor program in the vasculature and hematopoietic system. Tumor-derived factors downregulate Zbtb46 expression, leading to a pro-tumor TME. Importantly, the present disclosure shows that maintaining Zbtb46 expression in a therapeutic manner results in an anti-tumor TME and enhances the effectiveness of the immunotherapy/ICB treatment.

The present teachings show that the Zbtb46 transcription factor becomes downregulated in both dendritic and endothelial cells by tumor-produced factors. Zbtb46 downregulation activates endothelial cell and exuberant angiogenesis and suppresses dendritic cell generation by activating myeloid transcription factor CEBP-b and myeloid cell production. As such, the outcome of Zbtb46 downregulation leads to more robust tumor growth through both DC and EC arms. Systemic mRNA delivery using nanoparticles and anti-PD1 antibody treatment dramatically inhibited tumor growth and progression. As such, Zbtb46 mRNA delivery provides a unique means to promote the current immunotherapeutic approaches.

Tumor angiogenesis and immunity play critical roles in cancer progression and outcome. An inverse correlation of these two factors hints at common regulatory mechanism(s). Zbtb46, a repressive transcription factor and a widely accepted marker for classical dendritic cells (DCs), constitutes one such regulatory mechanism. Zbtb46 is downregulated in both DCs and endothelial cells (ECs) by tumor-derived factors to facilitate robust tumor growth. Zbtb46 downregulation leads to a stereotypical pro-tumor microenvironment (TME) characterized by dysfunctional vasculature and immunosuppressive cell accumulation. Analysis of cancer patient data revealed a similar association of low ZBTB46 expression with an immunosuppressive TME and a less favorable prognosis. In contrast, enforced Zbtb46 expression mitigates the pro-tumor TME features and restricts tumor growth.

Without being limited to any particular theory, Zbtb46-deficient ECs are mechanistically highly angiogenic, and Zbtb46-deficient bone-marrow progenitors upregulate Cebpb and divert the DC program to myeloid lineage output, potentially explaining the myeloid lineage skewing phenomenon in cancer. Conversely, enforced Zbtb46 expression normalizes tumor vessels and, by suppressing Cebpb, skews bone-marrow precursors towards more DC generation over macrophages, leading to an immune-hot TME. Remarkably, Zbtb46 mRNA treatment synergizes with anti-PD1 immunotherapy to improve tumor management in pre-clinical models. These findings identify Zbtb46 as a common regulatory mechanism for angiogenesis and for myeloid lineage skewing in cancer and suggest that maintaining its expression has therapeutic benefits.

Without being limited to any particular theory, tumor vessels and tumor immunity, two TME components, play influential roles in the tumor outcome. Tumor vessels are typically dilated, tortuous, irregular, and leaky, while tumor immunity is characterized by heightened immune-suppressive myeloid cell production over dendritic cell generation, known as myeloid lineage skewing. Previous studies have revealed an inverse correlation between tumor angiogenesis and tumor immunity. Although tumor vessel normalization improves tumor immunity and immune checkpoint blockade therapy outcomes, it is unclear how tumor vessel normalization leads to improved tumor immunity. As described herein, a common genetic mechanism directly controls both processes of these seemingly independent processes: tumor angiogenesis and tumor immunity. As disclosed herein, tumor angiogenesis and tumor immunity are coordinated by Zbtb46 in cancer.

AS described in the examines herein, Zbtb46 in mouse tumor models was downregulated in the ECs, DCs, and BM cells. Although Zbtb46 deficiency did not affect the vasculature or hematopoietic system in non-tumor-bearing mice, tumor-bearing ZKO mice supported more robust tumor growth, accompanied by higher microvascular density, reduced perfusion, augmented vascular leakage and hypoxia, decreased ratios of cytotoxic CTL/Treg and M1/M2 macrophages, diminished cDC1, and increased pro-tumor stroma. Enforcing Zbtb46 expression in the TME restricted tumor growth accompanied by a remodeling toward an immunostimulatory microenvironment, suggesting a tumor-suppressive role for Zbtb46 in the TME. An analysis of publicly available cancer patient datasets also showed that low ZBTB46 expression is associated with a worse prognosis. The BM chimera and conditional KO mouse data described in the examples herein suggested that both EC- and hematopoietic-specific Zbtb46 expression influence the regulation of tumor progression. Enforcing Zbtb46 expression leads to less angiogenic and more anti-tumor immune-stimulatory ECs, allowing for increased trans-endothelial leukocyte migration. Although Zbtb46 deficiency did not disrupt cDC1 antigen processing and presentation, as evidenced by the spontaneous rejection of the 1969 regressive sarcoma tumor model, 1956 progressive sarcoma-bearing ZKO mice had more granulocytic-monocytic progenitors and reduced pre-cDCs in both the BM and spleen. Overexpression of Zbtb46 in BM cells reversed this trend by generating more dendritic and fewer granulocytic cells, directly impacting tumor growth. Previous studies indicated that Zbtb46 targets and represses myeloid lineage-promoting transcription factors, such as Cebpb. The experimental resulted described herein confirmed that Cebpb is a direct target of Zbtb46, and Zbtb46's absence resulted in increased transcriptional activity of Cebpb. Mechanistically, enforcing Zbtb46 expression suppresses Cebpb transcriptional output and reduces myeloid lineage gene expression in BM cells.

Based on the results of the experiments described herein, Zbtb46 likely modulates DC vs. granulocytic lineage output and potentially explains how myeloid lineage skewing is achieved in cancer. Without being limited to any particular theory, the downregulation of Zbtb46 and consequent release of Cebpb repression leads to more generation of myeloid cells to meet the high demands of myeloid lineage production in emergency conditions, such as cancer.

Enforcing Zbtb46 expression promotes anti-tumor stromal and immune components in the TME, a prerequisite for effective ICB therapy. Indeed, Zbtb46 mRNA nanoparticles synergized with anti-PD1 treatment to control tumor growth and induce long-term remissions and immunological memory. The tumor-derived factors suppressed Zbtb46 expression in vitro, and pharmacological inhibition of these factors partially rescued the tumor-induced Zbtb46 suppression in vivo. Taken together, these results reveal a previously unknown tumor-suppressive role of Zbtb46 in the vascular and hematopoietic system. Zbtb46 maintenance was validated as an effective adjuvant therapy with immunotherapy in cancer management.

### Zbtb46 Modulation Agents

As described herein, Zbtb46 expression has been implicated in various diseases, disorders, and conditions. As such, modulation of Zbtb46 by any suitable means including, but not limited to, modulation of Zbtb46 expression with mRNA delivery, can be used for the treatment of such conditions. A Zbtb46 modulation agent can modulate Zbtb46 response including, but not limited to, inducing or inhibiting Zbtb46. Zbtb46 modulation can comprise modulating the expression of Zbtb46 in, on, and outside cells, modulating the quantity of cells that express Zbtb46, or modulating the quality of the Zbtb46-expressing cells.

Zbtb46 modulation agents can be any composition or method that can modulate Zbtb46 expression in cells including, but not limited to, delivery of Zbtb46 mRNA. By way of non-limiting example, a Zbtb46 modulation agent can be an activator, an inhibitor, an agonist, or an antagonist. As another example, the Zbtb46 modulation can be the result of gene editing.

A Zbtb46 modulation agent can be a Zbtb46 antibody (e.g., a monoclonal antibody to Zbtb46).

A Zbtb46 modulating agent can be an agent that induces or inhibits progenitor cell differentiation into Zbtb46-expressing cells (e.g., promoting Zbtb46-expressing cells in a tumor microenvironment). For example, Zbtb46 mRNA can be used to induce the expression of Zbtb46 in a tumor microenvironment.

Zbtb46 Signal Increase, Reduction, Elimination, or Inhibition by Small Molecule Inhibitors, shRNA, siRNA, or ASOs

As described herein, a Zbtb46 modulation agent can be used for use in cancer therapy. A Zbtb46 modulation agent can be used to reduce/eliminate or enhance/increase Zbtb46 signals. For example, a Zbtb46 modulation agent can be a small molecule enhancer or inhibitor of Zbtb46. As another example, a Zbtb46 modulation agent can be a short hairpin RNA (shRNA). As another example, a Zbtb46 modulation agent can be a short interfering RNA (siRNA).

As another example, RNA (e.g., long noncoding RNA (lncRNA)) can be targeted with antisense oligonucleotides (ASOs) as a therapeutic. As another example, Zbtb46 mRNA can be delivered to increase Zbtb46 expression in a tumor microenvironment. Processes for making ASOs targeted to RNAs, as well as mRNA delivery in general, are well known; see e.g. Zhou et al. 2016 Methods Mol Biol. 1402:199-213. Except as otherwise noted herein, therefore, the process of the present disclosure can be carried out in accordance with such processes.

### Zbtb46 Promoting Agent

One aspect of the present disclosure provides for targeting of Zbtb46, its receptor, or its downstream signaling. The present disclosure provides methods of treating or preventing immunotherapy-resistant cancer based on the discovery that Zbtb46 downregulation leads to an immunosuppressive pro-tumor microenvironment and enforced Zbtb46 expression leads to an immune hot tumor microenvironment.

As described herein, promoters of Zbtb46 (e.g., antibodies, fusion proteins, small molecules, DNA, RNA including mRNA) can reduce or prevent immunotherapy-resistant cancer. A Zbtb46 promoting agent can be any agent that can promote Zbtb46, upregulate Zbtb46, or overexpress Zbtb46.

As an example, a Zbtb46-promoting agent can promote Zbtb46-related signaling.

For example, the Zbtb46-promoting agent can be an antibody. As an example, the antibody can be an anti-Cebpb antibody or an anti-Zbtb46 antibody. Furthermore, the antibody can be a murine antibody, a humanized murine antibody, or a human antibody.

As another example, the Zbtb46 promoting agent can be an anti-Cebpb antibody, wherein the anti-Cebpb antibody prevents binding of Cebpb to its receptor or prevents activation of Cebpb and downstream signaling.

As another example, the Zbtb64 promoting agent can be an agent to suppress tumor-derived factors. For example, the Zbtb64 promoting agent can be an anti-inflammatory agent that leads to the increased expression of Zbtb46, wherein the anti-inflammatory agent prevents inhibition of Zbtb64 via downstream signaling.

As another example, the Zbtb46-promoting agent can be a fusion protein. For example, the fusion protein can be a decoy receptor for Zbtb46 or Cebpb. Furthermore, the fusion protein can comprise a mouse or human Fc antibody domain fused to the ectodomain of Zbtb46 or Cebpb.

As another example, a Zbtb46-promoting agent can be an mRNA, which has been shown to be a potent and specific promoter of Zbtb46 signaling.

As another example, a Zbtb46-promoting agent can be an excitatory protein that leads to the enhanced expression of Zbtb46. For example, the Zbtb46-promoting agent can be a viral protein, which has been shown to lead to Zbtb46 expression.

As another example, a Zbtb46-promoting agent can be a short hairpin RNA (shRNA) or a short interfering RNA (siRNA) targeting Zbtb46 or Cebpb.

As another example, a Zbtb46-promoting agent can be an sgRNA targeting Zbtb46 or Cebpb.

Methods for preparing a Zbtb46 promoting agent (e.g., an agent capable of promoting signaling) can comprise the construction of a protein/Ab scaffold containing the natural Zbtb46 receptor; developing promoters of the Zbtb46 receptor “down-stream”; or developing promoters of the Zbtb46 production “up-stream”.

Promoting Zbtb46 can be performed by genetically modifying Zbtb46 in a subject or genetically modifying a subject to increase or enforce expression of the Zbtb46 gene, such as through the use of CRISPR-Cas9 or analogous technologies, wherein, such modification reduces or prevents immunotherapy-resistant cancer.

### Chemical Agent:

Examples of Zbtb46 modulation agents are described herein.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O—CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, -CH2-cyclopropyl, -CH2-cyclobutyl, -CH2-cyclopentyl, -CH2-cyclopentadienyl, -CH2-cyclohexyl, -CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “UM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8)4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ Of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain | |
|---|---|
| Characteristic | Amino Acid |
| Aliphatic | G A P I L V |
| Non-polar | |
| Polar-uncharged | C S T M N Q |
| Polar-charged | D E K R |
| Aromatic | H F W Y |
| Other | N Q D E |

Conservative Substitutions II

| Side Chain | |
|---|---|
| Characteristic | |
| Non-polar | |
| (hydrophobic) | Amino Acid |
| A. Aliphatic: | A L I V P |
| B. Aromatic: | F W |
| C. Sulfur-: | M |
| containing | |
| D. Borderline: | G |
| Uncharged- | |
| polar | |
| A. Hydroxyl: | S T Y |
| B. Amides: | N Q |
| C. Sulfhydryl: | C |
| D. Borderline: | G |
| Positively | K R H |
| Charged | |
| (Basic): | |
| Negatively | D E |
| Charged | |
| (Acidic): | |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, |
| | Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, |
| | Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by any number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iTT RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, Zbtb46 signals can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate expression of Zbtb46 by genome editing can result in protection from autoimmune or inflammatory diseases, and promote a hot immune microenvironment in tumors.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)20NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for cancer therapy, particularly immunotherapy-resistant cancer, to target cells by the incorporation of Zbtb46 signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing cancer, in particular immunotherapy-resistant cancer, in a subject in need of administration of a therapeutically effective amount of a Zbtb46 modulation agent, so as to reverse immunotherapy-resistant cancer.

Methods described herein are generally performed on a subject in need thereof. A subject in need of the therapeutic methods described herein can be a subject having, diagnosed with, suspected of having, or at risk for developing immunotherapy-resistant cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a Zbtb46 modulation agent is, for example, an amount that would cause the desired therapeutic effect in a subject while minimizing undesired side effects. In various embodiments, an effective amount of a Zbtb46 modulation agent described herein can substantially inhibit immunotherapy-resistant cancer, slow the progress of immunotherapy-resistant cancer, or limit the development of immunotherapy-resistant cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a Zbtb46 modulation agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat immunotherapy-resistant cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a Zbtb46 modulation agent can occur as a single event or over a time course of treatment. For example, a Zbtb46 modulation agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for cancer.

A Zbtb46 modulation agent can be administered simultaneously or sequentially with another agent, such as an antibiotic, an anti-inflammatory, an immunotherapy such as anti-PD1 therapy, or another agent. For example, a Zbtb46 modulation agent can be administered simultaneously with another agent, such as an antibiotic, an anti-inflammatory, or an immunotherapy. Simultaneous administration can occur through administration of separate compositions, each containing one or more of a Zbtb46 modulation agent, an antibiotic, an anti-inflammatory, an immunotherapy, or another agent. Simultaneous administration can occur through administration of one composition containing two or more of a Zbtb46 modulation agent, an antibiotic, an anti-inflammatory, an immunotherapy, or another agent. A Zbtb46 modulation agent can be administered sequentially with an antibiotic, an anti-inflammatory, an immunotherapy, or another agent. For example, a Zbtb46 modulation agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character×log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character×log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a Zbtb46 modulation agent, solubilizing agents, salts, and other cancer drugs including immunotherapeutics. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1-Zbtb46 is a Negative Regulator of Tumor Progression

Background: To characterize the function of Zbtb46 disclosed herein, the following experiments were conducted. Endothelial cells (ECs) and classical dendritic cells (DCs) express Zbtb46 constitutively in physiological conditions. Using mouse cancer models, the direct involvement of Zbtb46 in solid tumor progression was investigated.

Methods: C57BL/6 mice were used as wild-type mice in this study. Zbtb46^{gfp/gfp} (Zbtb46 KO) mice were crossed with VEC-Cre, CD11c-Cre, and VAV-Cre mice (The Jackson Laboratory, Bar Harbor, ME) to generate VEC-Cre; Zbtb46^{gfp/gfp} conditional KO mice (EC-specific Zbtb46 KO), CD11c-Cre; Zbtb46^{gfp/gfp} conditional KO mice (DC-specific Zbtb46 KO), and VAV-Cre; Zbtb46^{gfp/gfp} conditional KO mice (Hematopoietic Zbtb46 KO), respectively. Zbtb46^{gfp/gfp} mice were crossed with MMTV-PyMT mice to generate Zbtb46 KO in the presence of MMTV-PyMT transgene (MMTV-PyMT Zbtb46^{gfp/gfp}). Littermate subjects were used as a control with the different knockout mice. Both male and female mice were used equally in any given experiment except in experiments utilizing both the genetic and orthotopic breast tumor models, where only female mice were used. The ages of the experimental animals were between 10 and 12 weeks.

MMTV-PyMT transgenic mice were utilized to generate a spontaneous model of breast cancer, where MMTV-LTR drives the expression of mouse mammary gland-specific polyomavirus middle T-antigen. Palpable tumors in the mammary gland of the MMTV-PyMT Zbtb46^{gfp/gfp} (Zbtb46 KO) mice were measured every week until 21 weeks of age to track the development and progression of tumorigenesis. Tumor volume was calculated by the equation, Volume=(largest diameter)×(smallest diameter)²×0.5.

Tumor transplantation studies were conducted by mixing 1 ml of growth factor reduced Matrigel (Cat: 354248; Corning) with 1 ml of cultured LLC-GFP tumor cell suspension (2×10⁶/ml in PBS); 100 ml of the mixture was subcutaneously injected into the back of the mice. 1956- and 1969-sarcoma cells were subcutaneously injected as 1×106 cells in 150 ml PBS+Matrigel solution (1:1) per mouse to the flank of the mice. PyMT-BO1 cells were orthotopically injected as 1×105 cells in 50 ml PBS+Matrigel solution (1:1) per mouse to the mammary fat pad of the mice. Palpable tumors started to develop 4-5 days after transplantation and tumor growth was measured until the end of the study. For overexpression studies, relevant lentiviral particles were intra-tumor injected as 15 ml/injection for a viral content of 2×10\hat{ }6 TU/injection, as many times as indicated in the relevant figures.

Bone marrow (BM) chimeric mice were generated as described herein. Wild-type recipient mice (CD45.1) were lethally irradiated with 950rad irradiation. Donor BM from the control (CD45.2) and Zbtb46 KO (CD45.2) mice were transplanted into the recipient mice retro-orbitally 24 hours post-irradiation. Flow cytometric analysis of the peripheral blood after five months of transplantation confirmed the successful BM reconstitution and generation of ‘hematopoietic Zbtb46 KO BM chimeric’ mice. Alternatively, lethally irradiated wild-type (CD45.2) and Zbtb46 KO (CD45.2) recipient mice received BM transplantation from wild-type (CD45.1) donors and generated ‘stromal Zbtb46 KO BM chimeric’ mice.

Arterial blood pressure, heart rate, and pressure-diameter measurements were collected as described. Zbtb46 KO and wild-type littermates were secured under 1.5% isoflurane anesthesia. A Millar pressure catheter (Cat: SPR-671, Millar, Inc.) was introduced to the ascending aorta, and heart rate, arterial systolic, diastolic, and mean blood pressures were recorded using the PowerLab data acquisition system (ADInstruments, Inc.). The ascending aorta and left common carotid artery of the mice were dissected and mounted on metal cannulae in a pressure myograph (Danish Myo Technology). Intravascular pressure was increased from 0 to 175 mmHg in 25 mmHg increments, and the vessel diameter was recorded at each pressure point. The average of 3 measurements at each pressure was reported.

RNA sequencing data from eighteen non-hematological TCGA tumor types were evaluated. Cancer types profiled included Adrenocortical carcinoma (ACC), Bladder Urothelial Carcinoma (BLCA), Breast invasive carcinoma (BRCA), Colon adenocarcinoma (COAD), Cervical squamous cell carcinoma and endocervical adenocarcinoma (CESC), Cholangiocarcinoma (CHOL), Head and Neck squamous cell carcinoma (HNSC), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), Prostate adenocarcinoma (PRAD), Rectum adenocarcinoma (READ), Skin Cutaneous Melanoma (SKCM), and Stomach adenocarcinoma (STAD). RNA sequencing data was downloaded from the GDC pan cancer portal (https://gdc.cancer.gov/about-data/publications/pancanatlas). Data was processed using the Firehose pipeline with upper quantile normalization. The primary tumor sample was favored for patients with more than one RNA-seq sample. xCell, a gene signatures-based enrichment approach, was used to delineate the enrichment of 64 immune and stromal cell types. The xCell R package was used to generate raw enrichment scores, transform the raw enrichment scores into linearly scaled scores, and apply a spillover compensation to derive corrected enrichment scores. The distribution of enrichment scores for patients with high (>50%) and low (<50%) levels of ZBTB46 expression were compared. Mann-Whitney test was used to calculate statistical significance with an alpha value of 0.05.

For survival analysis, the Pathology section of The Human Protein Atlas was accessed through its website (https://www.proteinatlas.org/) and searched for ZBTB46 gene expression in different tumor types along with the clinical outcome. Briefly, The Cancer Genome Atlas (TCGA) project of Genomic Data Commons (GDC) collects and analyzes multiple human cancer samples. RNA-seq data from 17 cancer types representing 21 cancer subtypes with a corresponding major cancer type in the Human Pathology Atlas were included to allow for comparisons between the protein staining data from the Human Protein Atlas and RNA-seq from TCGA data. The TCGA RNA-seq data was mapped using the Ensembl gene id available from TCGA, and the FPKMs (number Fragments Per Kilobase of exon per Million reads) for each gene were subsequently used for quantification of expression with a detection threshold of 1 FPKM. Based on the FPKM value of each gene, patients were classified into two expression groups, and the correlation between expression level and patient survival was examined. The prognosis of each group of patients was examined by Kaplan-Meier survival estimators, and the survival outcomes of the two groups were compared by log-rank tests.

For flow cytometry analysis, bone marrow (BM) was isolated from the experimental animals by flushing the tibia and femur. The spleen was collected and meshed into a single-cell suspension using the back side of a sterile 5 ml syringe plunger. Peripheral blood (PB) was collected by intra-cardiac puncture from the sacrificed animals immediately and processed for making single-cell suspensions following standard procedure. Lungs were minced and processed by standard enzymatic digestion consisting of Collagenase-I (Cat: LS004194, Worthington). Tumor-draining lymph nodes were isolated, disrupted, and digested with Collagenase-IV (Cat: LS004186, Worthington). Tumor tissues were harvested, minced into fine pieces, and dissociated into single-cell suspensions with an enzymatic digestion buffer consisting of Collagenase-II (for subcutaneous tumors) (Cat: LS004176, Worthington) or Collagenase-III (for breast tumors) (Cat: LS004182, Worthington), along with Dispase-II (Cat: D4693, Millipore Sigma) and Deoxyribonuclease 1 (Cat: LS002139, Worthington). Next, the cell suspensions were incubated with LIVE/DEAD™ Fixable Blue Dead Cell Stain Kit (Cat: L34961) along with different panels of fluorophore-conjugated surface staining antibodies. For subsequent intracellular staining, cell suspensions were fixed and permeabilized using either Foxp3/Transcription Factor Staining Buffer Set (Cat: 00-5523-00, ThermoFisher Scientific) or Intracellular Fixation & Permeabilization Buffer Set (Cat: 88-8824-00, ThermoFisher Scientific) and subsequently stained with intracellular antibodies. Samples were analyzed using either BD LSRFortessa™ X-20 (BD Biosciences) or BD FACSymphony™ A3 (BD Biosciences) and later processed with FlowJo software (BD Life Sciences). CD45-CD31+ECs and CD45+CD11c+MHCII+cDCs were FACS sorted using BD FACSAria-II (BD Bioscience). Sorted cells were purity tested by secondary flow cytometry run and later processed for downstream applications total RNA isolation using RNeasy mini kit (Cat: 74104, Qiagen) by following the manufacturer's instructions. Markers used for different cell lineages are given below:

| Cell Type | Markers |
|---|---|
| Lineage (Lin) | CD3+Ter119+B220+Gr1+ |
| MkP | Lin−Sca1−cKit+CD41+CD150+ |
| GKP | Lin−Sca1−cKit+CD41−CD150−CD16/32+ |
| Pre-cDC | Lin−Sca1−IL7R−MHCII−CD16/32−CD11c+cKit−CD135+ |
| CMP | Lin−Sca1−IL7R−MHCII−CD16/32−CD11c−CD41−CD135+cKit hi |
| CDP | Lin−Sca1−IL7R−MHCII−CD16/32−CD11c−CD41−CD135+cKit int |
| cDC | CD45+CD11c+MHCII+ |
| cDC1 | CD45+CD11c+MHCII+Xcr1+ |
| CTL | CD45+CD3+CD8+ |
| Treg | CD45+CD3+CD4+CD25+Foxp3+ |
| M1 | CD45+CD11b+F4/80+iNOS+ |
| M2 | CD45+CD11b+F4/80+CD206+CX3CR1+ |
| B | CD45+B220+ |
| NK | CD45+NK1.1+ |
| EC | CD31+CD45− |

Immunofluorescence studies were performed as described herein. Harvested tumors were thinly sliced and fixed in 10% buffered formalin (Cat: 16004-112, VWR), immersed in 30% (w/v) sucrose solution for 48 hours to cryo-protect the tissue, frozen in NEG-50 frozen section medium (Cat: 6502, ThermoFisher Scientific) using liquid nitrogen and 2-methyl butane system, and sectioned in 8 mm thickness using a Leica Cryostat microtome (Cat: CM1850, Germany). Afterward, tissue sections were blocked using freshly made blocking buffer (3% essentially IgG-free BSA (Cat: A9085), 0.3% Triton X-100 (Cat: X100), and Fc blocker (Cat: 101301)). Next, sections were incubated with different primary antibodies for 16 h at 4° C., followed by visualization with appropriate secondary antibodies. Finally, the sections were counterstained for nuclei with DAPI, cured with ProLong Diamond Antifade mountant, and sealed with nail polish for preservation. To detect intra-tumoral hypoxia in mice, Hypoxyprobe-1 solution (Cat: HP6-100Kit) was intraperitoneally administered 90 minutes prior to sacrifice as 100 mg/Kg bodyweight. FITC-conjugated anti-pimonidazole mouse IgG1 monoclonal antibody was used to detect the extent of hypoxia. For the vascular leakage and perfusion experiments, FITC-conjugated 70KD dextran (60 mg/Kg bodyweight) (Cat: 46945) and FITC-conjugated lectin (8 mg/Kg bodyweight) (Cat: L9381), respectively, were injected through tail vein 15 minutes prior to sacrifice. Hamster anti-mouse CD31 (Clone: 2H8, Cat: MA3105) was used as the primary antibody. AF568 goat anti-hamster (Cat: A-21112) was utilized as the secondary antibody. The sections were examined using the Olympus Fluoview 1200 confocal microscope system and minimally processed with Imaris (Bitplane) software. At least five pictures from every section were processed using ImageJ software (NIH) for quantification purposes.

LLC-GFP cells, PyMT-BO1, and HEK293T cells were cultured in DMEM high glucose growth medium supplemented with 10% (v/v) FBS, 100 unit/ml penicillin-streptomycin. 1956 and 1969 sarcoma cells were cultured in RPMI 1640 growth medium supplemented with 10% (v/v) FBS, 100 unit/ml penicillin-streptomycin, 1% (v/v) L-glutamine (200 mM), 1% (v/v) Sodium Pyruvate (100 mM), 0.5% (v/v) Sodium Bicarbonate (7.5% w/v stock), and 0.1% (v/v) 2-Mercaptoethanol. All MCEC cell lines were maintained in M199 growth medium, supplemented with 20% (v/v) FBS, 100 units/ml penicillin-streptomycin, and 10 mM HEPES. In bone marrow cell-related experiments, bone marrow cells were cultured in Iscove's Modified Dulbecco's media supplemented with 10% (v/v) FBS, 100 unit/ml penicillin-streptomycin.

Lentiviral shRNA and overexpression particle production was conducted with pLKpuro lentiviral mouse Zbtb46 shRNA clones TRCN0000125839 (NM_028125.1-2420s1c1), TRCN0000125840 (NM_028125.1-364s1c1), TRCN0000125841 (NM_028125.1-1358s1c1), TRCN0000125842 (NM_028125.1-321s1c1), and TRCN0000125843 (NM_028125.1-1231s1c1) and mouse Cebpb shRNA clones TRCN0000231407 (NM_009883), TRCN0000231408 (NM_009883), TRCN0000231410 (NM_009883), TRCN0000231411 (NM_009883), and TRCN0000231409 (NM_009883) were purchased from Millipore Sigma. HEK293T cells were transfected with the mentioned shRNA clones or pCSII-EF1-Zbtb46-IRES2-Bsr or pCSII-EF1-Cebpb-IRES2-Bsr constructs along with pCAG-HIVgp and pCMV-VSV-G-RSV-Rev (with a ratio of 4:3:1) by using Calcium Phosphate method. Sixteen hours after transfection, the media was changed, and cells were grown for an additional 48 hours. Subsequently, the supernatant was harvested and concentrated by Lenti-X-Concentrator (Cat: 631232, Clontech). The virus titer was determined using the Lenti-X™ p24 Rapid Titer Kit (Cat: 632200, Clontech).

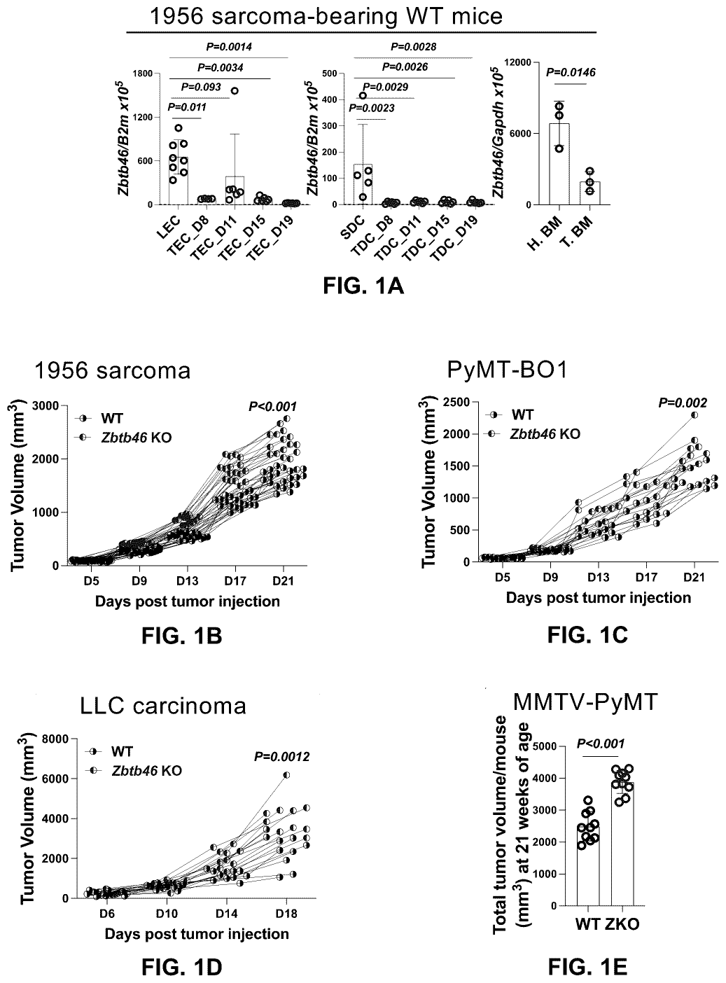

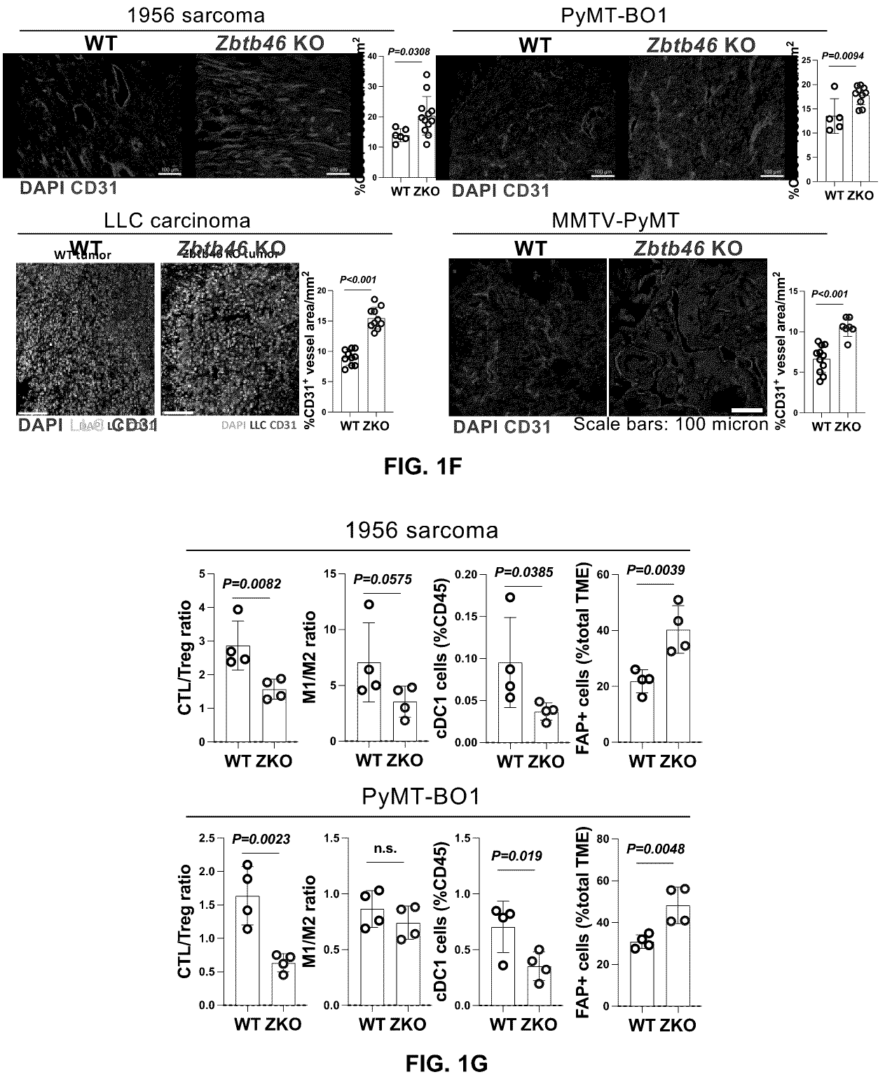

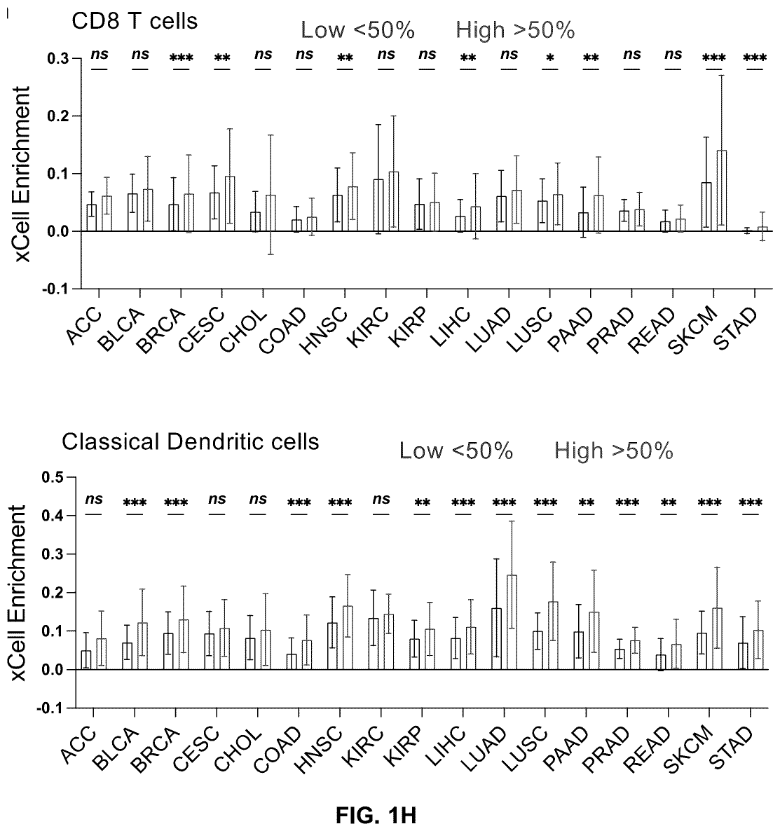

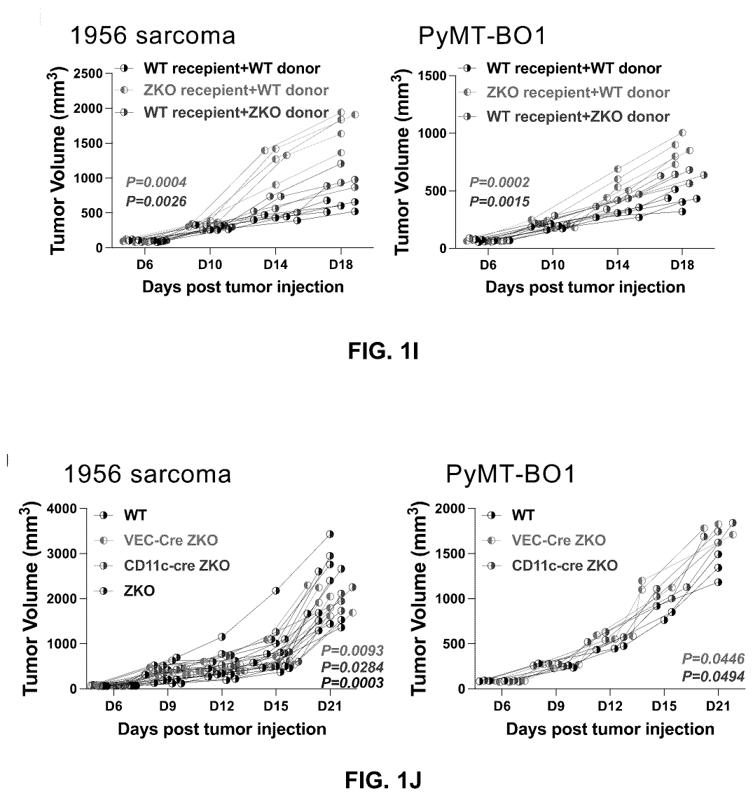

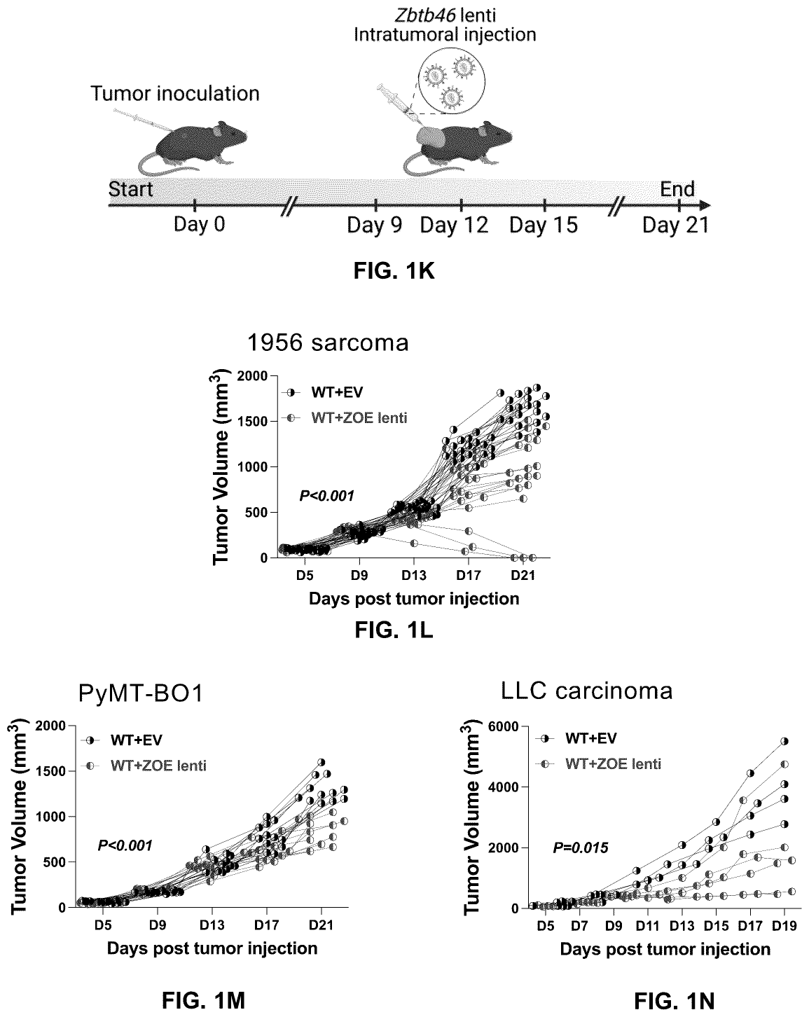

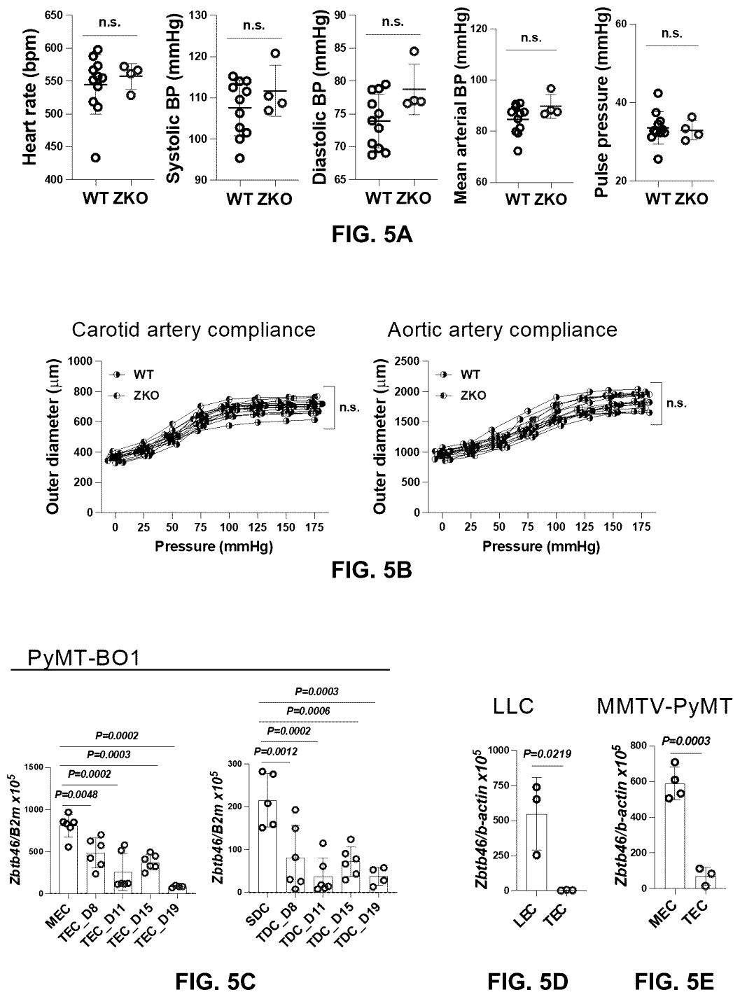

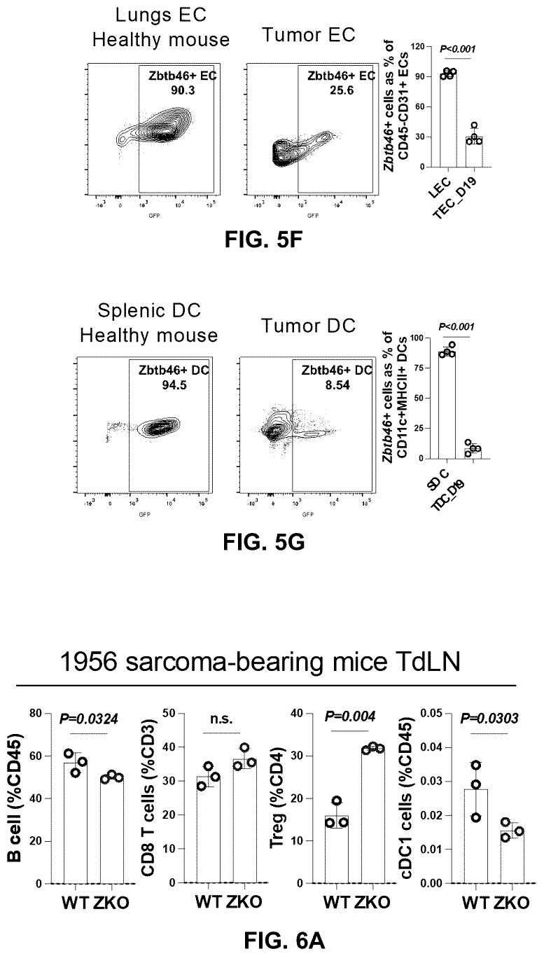

Results: Zbtb46 expression was downregulated in the tumor-associated ECs and DCs in the wild-type (WT) mice bearing 1956 sarcoma, LLC carcinoma, and PyMT-BO1 (orthotopic) and MMTV-PyMT (genetic) breast cancers (FIG. 1A, FIG. 5C, FIG. 5D, FIG. 5E, FIG. 5F, FIG. 5G) and in the bone marrow (BM) cells (FIG. 1A).

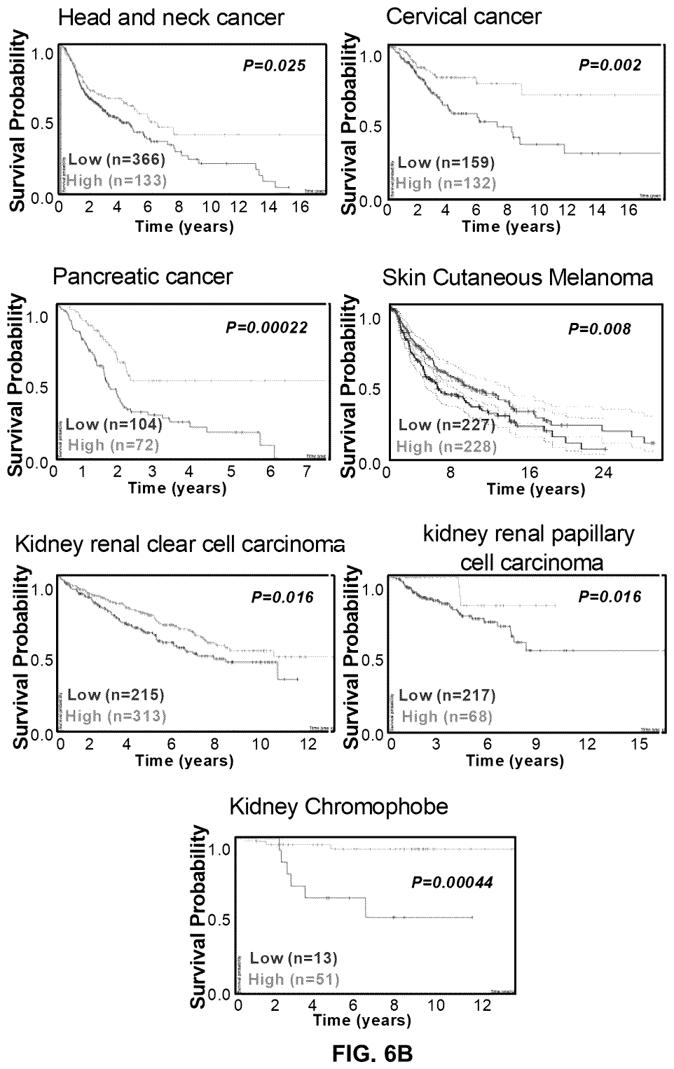

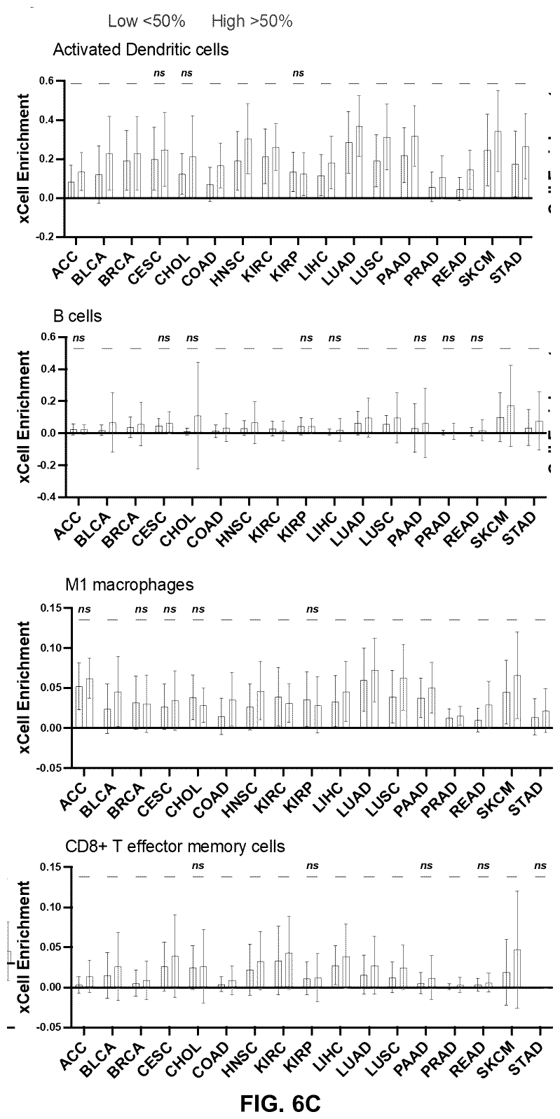

Zbtb46 knock-out (ZKO) mice were challenged with the same tumor models to determine whether this suppression contributes to tumor outcome. Compared to their WT counterparts, ZKO mice exhibited more robust tumor growth (FIG. 1B, FIG. 1C, FIG. 1D, FIG. 1E). Further analysis revealed enhanced pro-tumor microenvironment (TME) features in the ZKO mice, including exuberant angiogenesis, lower CTL/Treg and M1/M2 ratios, decreased cDC1 presence, and increased fibroblast activation (FIG. 1F, FIG. 1G, FIG. 6A). Analysis of the cancer patient survival data showed that a lower expression of ZBTB46 is associated with a worse prognosis in multiple cancer types (FIG. 6B). Similar to the animal models, patients with a lower expression of ZBTB46 had reduced anti-tumor immune components (FIG. 1H, FIG. 6C). Together, these data suggest a constitutive inhibitory role for Zbtb46 in tumor growth.

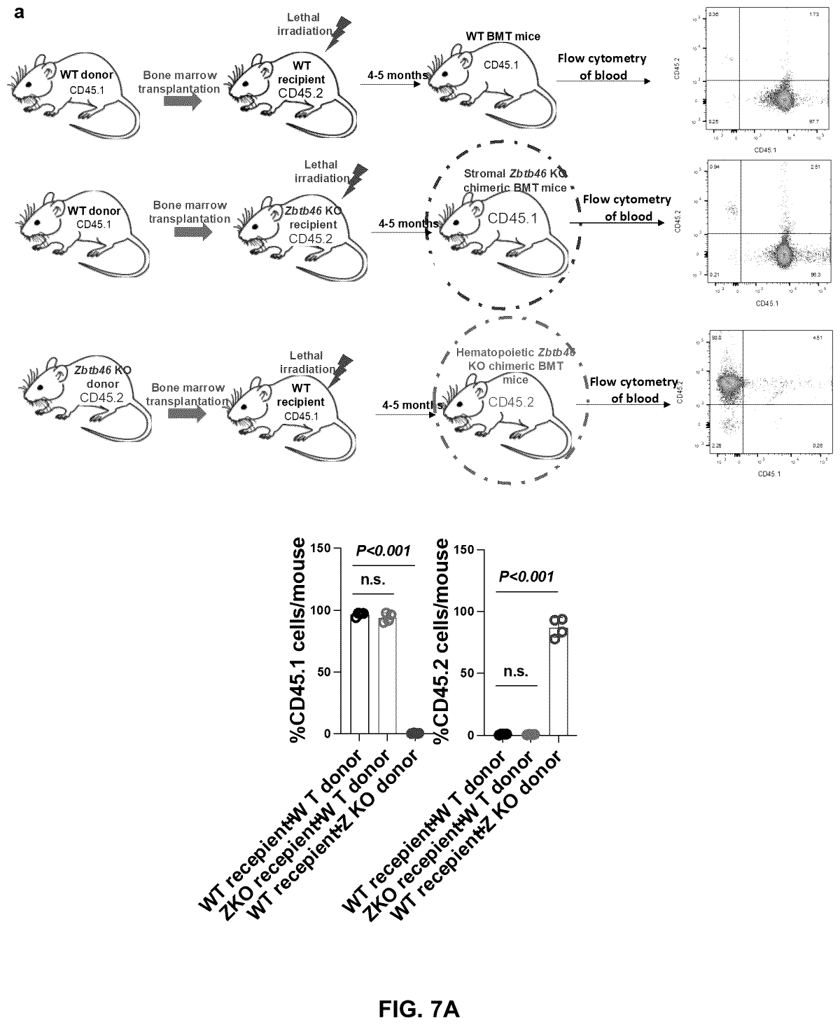

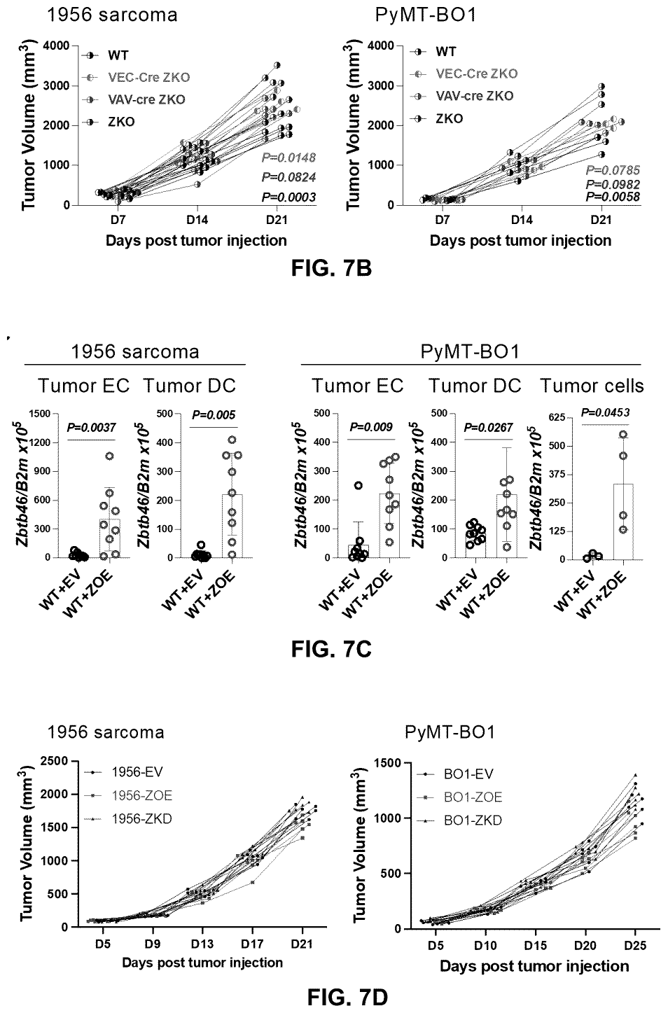

We examined the relative contribution of EC- and DC-specific Zbtb46 expression in the tumor growth by employing endothelial (VEC-Cre)- or hematopoietic (CD11c-Cre or VAV-Cre)-specific deletion of Zbtb46. BM chimeras were generated for the same purpose (FIG. 7A). In both systems, it was observed that Zbtb46 was required in both the endothelial and hematopoietic cells for optimal tumor control (FIG. 1I, FIG. 1J, FIG. 7B). The most robust tumor growth was observed in the global ZKO mice, suggesting a potential collaborative role for the EC- and DC-specific Zbtb46 expression in suppressing tumor growth.

Enforced expression of Zbtb46 was investigated to determine if Zbtb46 could halt tumor progression. Intra-tumor lentiviral delivery of Zbtb46 led to sustained expression in the TME and reduced tumor mass (FIG. 1K, FIG. 1L, FIG. 1M, FIG. 1N, FIG. 7C). Importantly, Zbtb46 overexpressing tumor cell lines did not have much growth difference compared to the parental tumor cell lines, suggesting a TME-centric role for Zbtb46 expression in suppressing tumor growth (FIG. 7D).

### Example 2-Zbtb46 Normalizes Tumor Vasculature

Background: To investigate the role of Zbtb46 in tumor vascularization, the following experiments were conducted. Tumor vessels are inherently abnormal, featuring impaired vascular perfusion and excessive leakage that leads to a hypoxic and immunosuppressive microenvironment.

Methods: RNA sequencing data from WT and Zbtb46 overexpressing MCECs were pre-processed and analyzed for differentially expressed genes using edgeR. Genes with fewer than five counts in at least three samples were filtered out. Count data was normalized to account for varying library sizes. Normalized data was fit to a negative binomial generalized log-linear model, and differentially expressed genes were extracted. In-built functions for gene ontology analysis were used to identify GO terms enriched in WT and Zbtb46 overexpression MCECs. Sequencing data is available as GSE226087, the contents of which are incorporated herein in its entirety.

Tube formation assay was performed by overnight serum-starving endothelial cells, which were then plated on Matrigel (Cat: 96992, Corning) coated 24-well plates (3×104 cells/well) and incubated at 37° C. for 6 hours before taking pictures with a Leica DFC 310 FX microscope system as described before. The angiogenesis analyzer module of ImageJ software (NIH) was used to quantify the total tube length, number of loops, and number of branches.

The T cell trans-endothelial migration assay is described herein. T cell migration potential across an endothelial barrier in vitro was assessed using a QCM™ Leukocyte Trans-endothelial Migration Colorimetric Assay kit (Cat: ECM557, Millipore Sigma) following the manufacturer's instruction. T cells used in the study were isolated from the spleens harvested from 1956 sarcoma subcutaneous tumor-bearing wild-type mice with the APC CD45 (Cat: 103112, Clone: 30-F11), PE CD3 (Cat: 100206, Clone: 17A2), BV650 CD4 (Cat: 100469, Clone: GK1.5), PerCP/Cy5.5 CD8a (Cat: 100734, Clone: 53-6.7). Cytotoxic T cells were sorted using BD FACSAria™ II (BD Biosciences) as CD45+CD3+CD4-CD8+ population. Endothelial monolayers were treated with either TNFa (20 ng/ml) for 12 hours and washed with PBS before placing the harvested T cells on the endothelial monolayer. The relative abundance of migrated T cells was calculated by measuring the absorption of the samples at 450 nm following the WST-1 reagent staining.

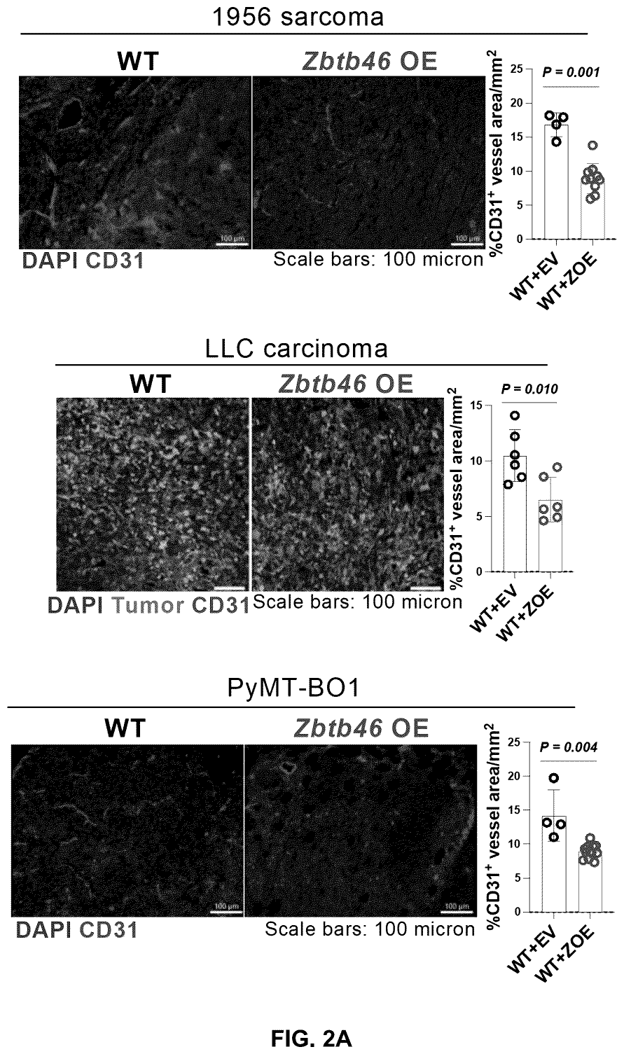

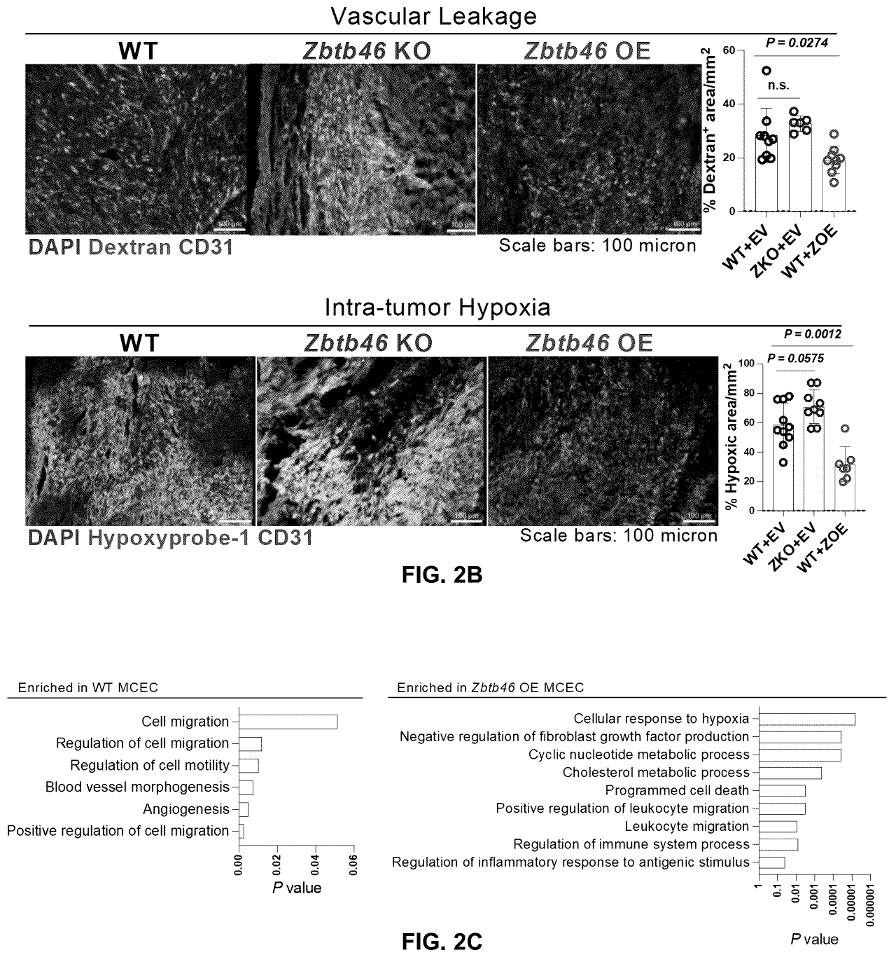

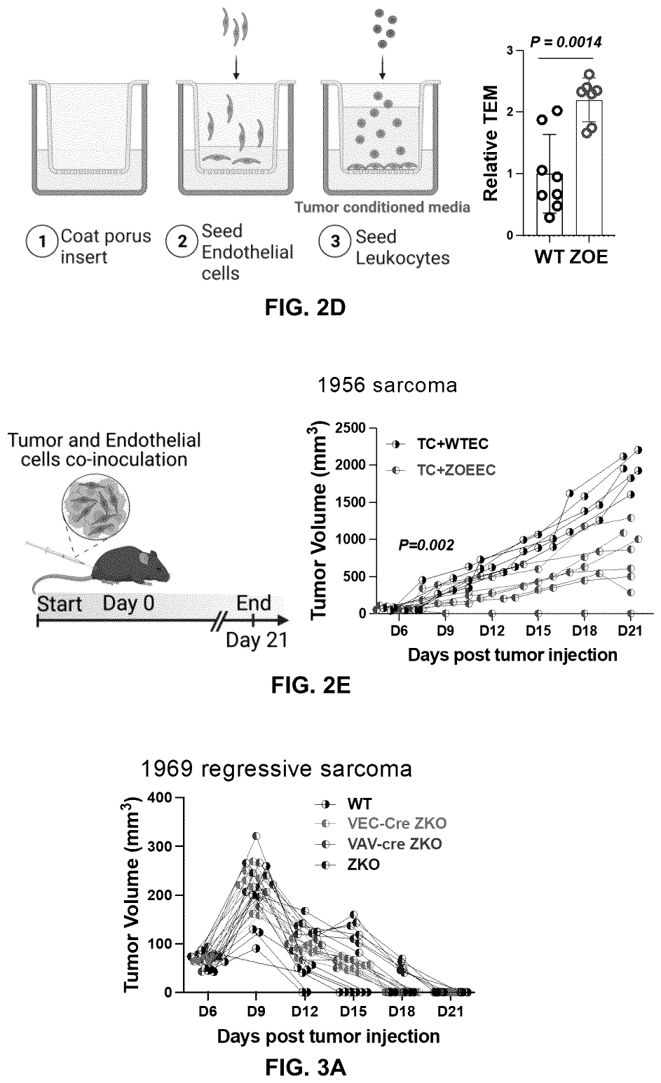

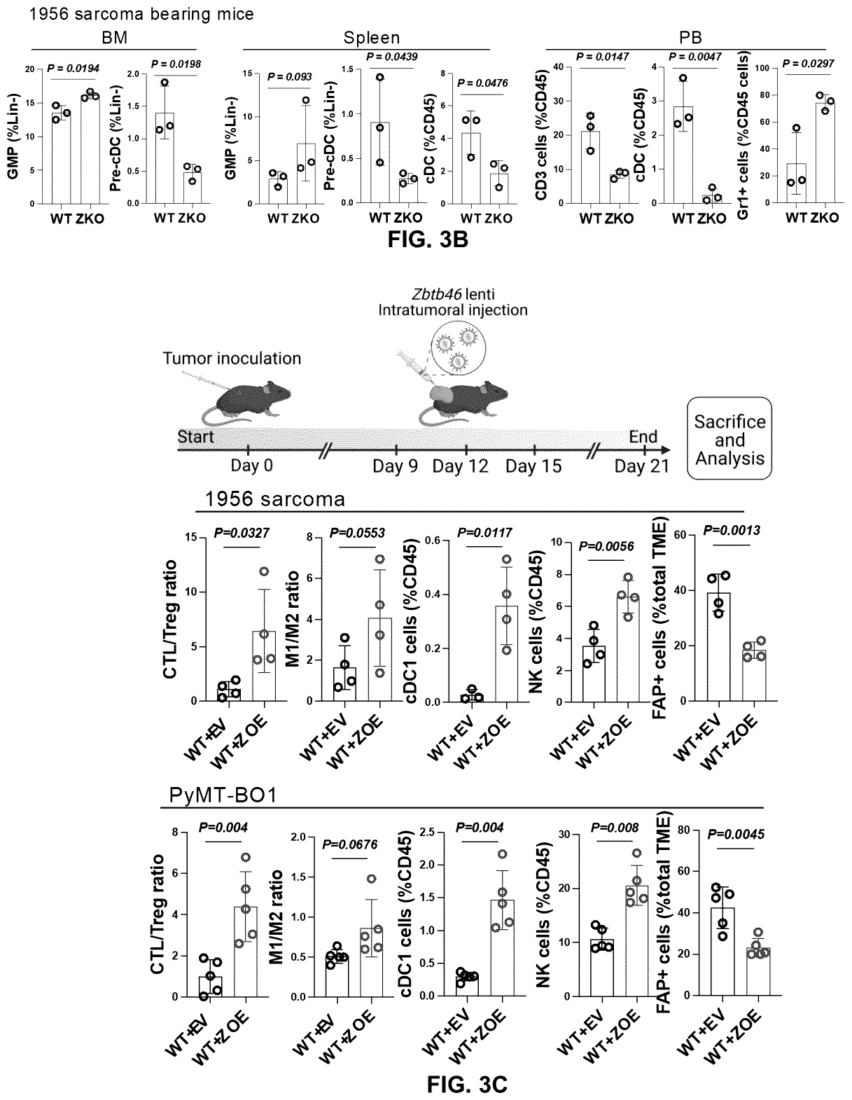

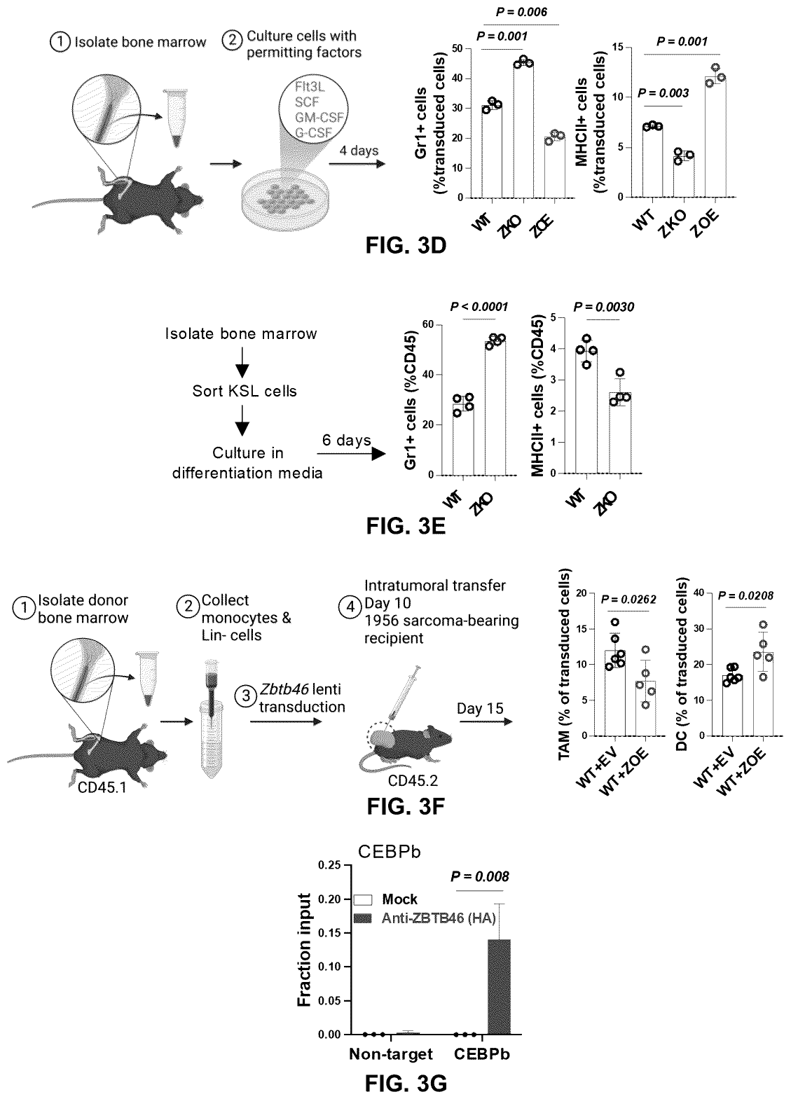

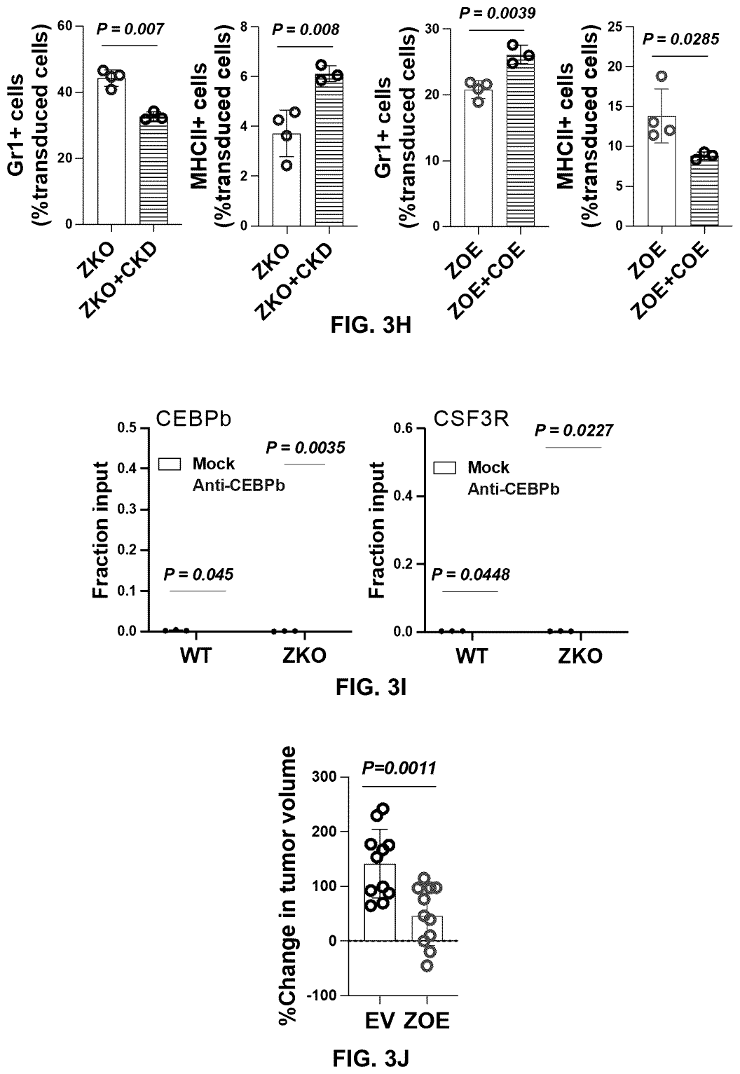

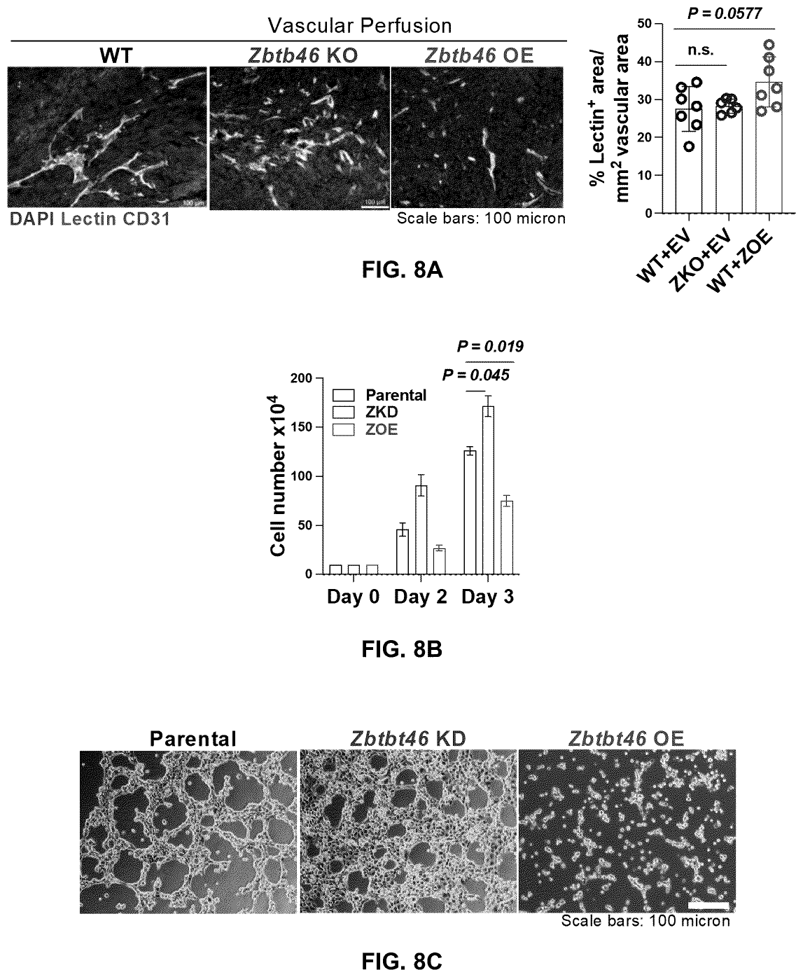

Results: Along with the tumor growth restriction, intra-tumor lentiviral Zbtb46 expression curbed tumor angiogenesis (FIG. 2A). While tumor vasculature was more dysfunctional in the ZKO mice, intra-tumor enforced Zbtb46 expression improved all the functional characteristics leading to vascular normalization (FIG. 2B, FIG. 8A). Normalized tumor vessels are known to support more anti-tumor immune and stromal TME, which could partly explain the decreased anti-tumor immune components observed in the ZKO mice and the opposite following the enforced Zbtb46 expression (FIG. 1G, FIG. 3C).

Zbtb46 overexpression in mouse cardiac endothelial cells (MCEC) made them quiescent, as evidenced by modestly reduced proliferation and significantly limited tube-formation capabilities in vitro (FIG. 8B, FIG. 8C). Bulk mRNA sequencing revealed that while the parental MCEC cells were enriched in migration and angiogenic pathways, Zbtb46 overexpressing MCEC cells were enriched in hypoxic stress response and immune-supportive pathways (FIG. 2C). Zbtb46 overexpressing MCEC cells allowed more leukocyte trans-endothelial migration, another hallmark feature of the normalized vasculature (FIG. 2D). We established a co-transplantation system consisting of ECs and tumor cells, where the co-transplanted ECs directly impact tumor growth. EC-specific Zbtb46 overexpression significantly restricted the tumor progression, solidifying the importance of Zbtb46 expression in the tumor vasculature (FIG. 2E).

### Example 3-Zbtb46 Promotes Dc Generation while Restricting Myeloid Lineage Output

Background: To investigate the role of Zbtb46 in DC function in a cancer environment, the following experiments were conducted. Zbtb46 deficiency does not compromise classical DC function in a steady state. To assess whether the scenario is different in cancer, the global, EC-specific (VEC-Cre), and hematopoietic-specific (CD11c-Cre or VAV-Cre) ZKO mice were challenged with 1969 sarcoma tumors that regress spontaneously in immune-competent WT mice.

Methods: The Zbtb46 mRNA-p5RHH peptide nanoparticle was prepared by using the Zbtb46 ORF sequence, which was made into a modified mRNA transcript with complete substitution of pseudo-U in RNase-free water from TriLink Biotechnologies. CleanCap® EGFP mRNA (Cat: L-7201-100) was used as mRNA control. 8 ml (1 mg/ml) of the mRNA solution was mixed with 5 ml of 20 mM p5RHH peptide solution and 187 ml of 1×HBSS (Gibco) to prepare the nanoparticle complex and immediately injected into the mouse through the tail vein.

Bone marrow cell differentiation was performed by using total bone marrow cells which were harvested by flushing out the tibia and femur of the experimental animals. Cells were transfected with different lentiviral particles by spin infection at 800×g for 30 min at 32° C. in the presence of 2 μg/ml polybrene. Cells were later cultured in complete IMDM media with 10 ng/ml of Flt3L (Cat: 250-31L, Peprotech), stem cell factor (SCF) (Cat: 250-03, Peprotech), GM-CSF (Cat: 315-03, Peprotech), and G-CSF (Cat: 250-05, Peprotech). Cells were harvested and analyzed by FACS after four days of culture.

The KSL (Lin− cKit+ Sca1+) cell differentiation assay is described herein. Total bone marrow cells were harvested by flushing out the tibia and femur of the experimental animals. Cells were then stained with PE/Cy7 conjugated anti-Gr-1 (RB6-8C5), -CD11b (M1/70), -B220 (RA3 6B2), -Ter119 (TER-119) and -CD3 (145-2C11), in combination with APC/Cy7 c-Kit (2B8), PerCp/Cy5.5 Sca1 (E13-161.7), PE CD34 (RAM34) and BV421 CD16/32 antibodies for 40 min on ice. KSL cells (Lin− cKit+ Sca1+) were sorted on FACS Aria II (BD Biosciences) sorter using 85 μm nozzles directly into a round-bottom 96-well plate at a density of 1000 cells/well. Culture media consisted of StemSpan serum-free base medium (StemCell Technologies), 10% FBS, penicillin (50 U/mL) and streptomycin (50 U/mL), SCF (25 ng/ml, PeproTech), FLT3L (20 ng/ml, PeproTech), IL3 (1% supernatant), mTPO (20 ng/ml, PeproTech), IL6 (10 ng/ml, PeproTech), IL11 (10 ng/ml, PeproTech), M-CSF (10 ng/ml, PeproTech), G-CSF (10 ng/ml, PeproTech), and GM-CSF (10 ng/ml, PeproTech). Cells were harvested and analyzed by FACS after six days of culture.

Bone marrow monocyte enrichment was performed by harvesting the tibia and femur of the experimental animals, the bone marrow was flushed, and later monocytes were enriched from the total bone marrow cells using the Monocyte Isolation Kit (BM) from Miltenyi Biotec (Cat: 130-100-629) through a negative selection process following the manufacturer's instruction. This enriched population contained mostly monocytes and precursor lineage-negative cells.

Chromatin Immunoprecipitation (ChIP)-qPCR was performed using enriched monocyte and lineage-negative cells from bone marrow, which were cross-linked with 1% formaldehyde and lysed with cell membrane lysis buffer, followed by further incubation with nuclear membrane lysis buffer. For the Zbtb46 overexpression system, before proceeding to lysate preparation, after the isolation of the enriched population, Zbtb46-HA overexpression lentiviral particles were used to transduce the cells by spin infection at 800×g for 30 min at 32° C. in the presence of 2 μg/ml polybrene and then incubated at 37° C. for 24 hours in complete IMDM media. The lysate was incubated with either an anti-CEBPb antibody (Cat: 23431-1-AP, Proteintech) or anti-HA antibody (Cat: ab9110, Abcam) followed by protein A/G sepharose beads (Cat: sc-2002, Santa Cruz Biotechnology). The beads were washed to isolate immunoprecipitated DNA fragments that were later subjected to qPCR. For the anti-ZBTB46 experiment Cebpb peak-1 enrichment (DNA location chr20:50189053-50189756; corresponding mouse region: chr2:167687660-167688195) was assessed by qPCR using primer-set forward sequence CCCCAGCTCAGCAGATAACA (SEQ_ID_NO: 1) and reverse sequence AGGCTTCTCAGGTGATTGCG (SEQ_ID_NO: 2). For the anti-CEBPb experiment, Cebpb peak-1 enrichment (DNA location chr2:167687679-167688023) was assessed by qPCR with primer-set forward sequence AAGGGCACAGGGAGATGTCA (SEQ_ID_NO: 3) and reverse sequence GGTGTTGCTCAACCTTCGGT (SEQ_ID_NO_4); Csf3r peak-6 enrichment (DNA location chr4:126017669-126017995) was assessed by qPCR with primer-set forward sequence GACAACGCTGGCACTTTTGTA (SEQ_ID_NO: 5) and reverse sequence TGTGCAAGCAGGTCATTGTG (SEQ_ID_NO: 6).

Intra-tumoral transfer of enriched bone marrow monocytes. Enriched monocytes and precursor lineage-negative cells from CD45.1 wild-type donor mice were transduced with either empty vector-mCherry or Zbtb46-mCherry lentiviral particles by spin infection at 800×g for 30 min at 32° C. in the presence of 2 μg/ml polybrene. Later, the cells were resuspended in PBS as 1×105 cells/50 μl and injected intra-tumor into the day 10 post-transplant 1956 sarcoma tumor-bearing CD45.2 wild-type recipient mice. Tumor volumes were measured periodically and harvested at day 15 post-transplantation for flow cytometric analysis to track donor-derived (CD45.1+) transduced (mCherry+) cell polarization into either macrophage or dendritic cells.

Quantitative real-time reverse transcription PCR (qRT-PCR) was performed. cDNA was prepared with qScript™ CDNA SuperMix (Cat: 101414-106, VWR) according to the manufacturer's protocol. Gene expression was measured by quantitative real-time RT-PCR using primers. The sequences of various forward and reverse primers are summarized in Table 1 below.

TABLE 1
qRT-PCR Primers

| Gene | Forward Sequence | Reverse Sequence |
|---|---|---|
| Zbtb46 | ATCACTTCTCACTACCGGCA | AAGACGTTCTTATGTGCCTT |
| | T (SEQ ID NO: 7) | GAA (SEQ ID NO: 8) |
| Cebpb | CGCCTTATAAACCTCCCGCT | TGGCCACTTCCATGGGTCTA |
| | (SEQ ID NO: 9) | (SEQ ID NO: 10) |
| Cd5l | GATCGTGTTTTTCAGAGTCT | TGCAGTCAACCCCTTGAATA |
| | CCA (SEQ ID NO: 11) | AG (SEQ ID NO: 12) |
| Runx3 | CAGGTTCAACGACCTTCGAT | GTGGTAGGTAGCCACTTGGG |
| | T (SEQ ID NO: 13) | (SEQ ID NO: 14) |
| Irf4 | TCCGACAGTGGTTGATCGAC | CCTCACGATTGTAGTCCTGC |
| | (SEQ ID NO: 15) | TT (SEQ ID NO: 16) |
| Ifi44 | AACTGACTGCTCGCAATAAT | GTAACACAGCAATGCCTCTT |
| | GT (SEQ ID NO: 17) | GT (SEQ ID NO: 18) |
| Itgax | CTGGATAGCCTTTCTTCTGC | GCACACTGTGTCCGAACTCA |
| | TG (SEQ ID NO: 19) | (SEQ ID NO: 20) |
| Ccr7 | TGTACGAGTCGGTGTGCTTC | GGTAGGTATCCGTCATGGTC |
| | (SEQ ID NO: 21) | TTG (SEQ ID NO: 22) |
| Lifr | TACGTCGGCAGACTCGATAT | TGGGCGTATCTCTCTCTCCT |
| | T (SEQ ID NO: 23) | T (SEQ ID NO: 24) |
| Mertk | CAGGGCCTTTACCAGGGAGA | TGTGTGCTGGATGTGATCTT |
| | (SEQ ID NO: 25) | C (SEQ ID NO: 26) |
| Mafb | TTCGACCTTCTCAAGTTCGA | TCGAGATGGGTCTTCGGTTC |
| | CG (SEQ ID NO: 27) | A (SEQ ID NO: 28) |
| Vegfa | CTGCCGTCCGATTGAGACC | CCCCTCCTTGTACCACTGTC |
| | (SEQ ID NO: 29) | (SEQ ID NO: 30) |
| Csf3r | CTGATCTTCTTGCTACTCCC | GGTGTAGTTCAAGTGAGGCA |
| | CA (SEQ ID NO: 31) | G (SEQ ID NO: 32) |

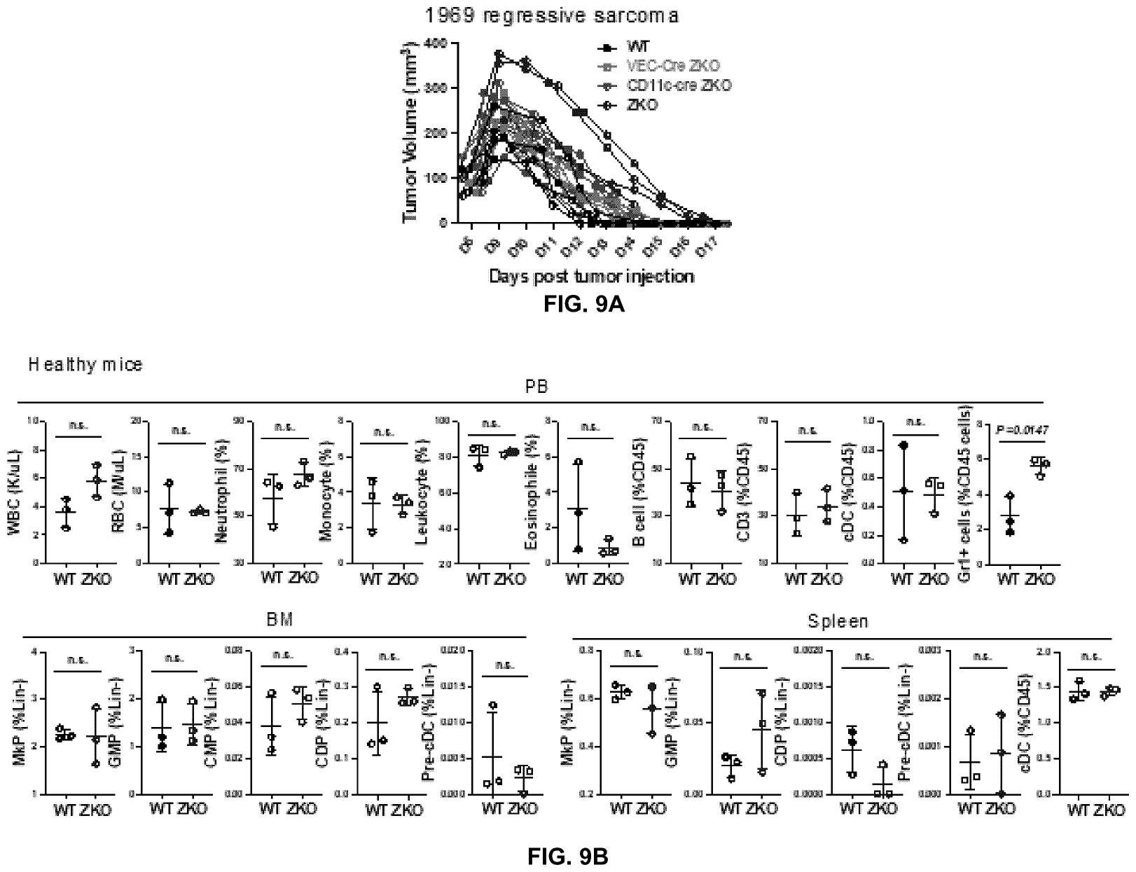

Results: All of the ZKO mice rejected the inoculated 1969 tumor mass, albeit with a slightly delayed kinetics compared to the WT mice (FIG. 3A, FIG. 9A). This outcome is unlike what was reported for Batf3 KO mice, a gene critical for cDC1 lineage development and cDC1-mediated cross-presentation for tumor rejection, suggesting an absence of any direct involvement of Zbtb46 in classical DC function.

To determine if there might be any other immune system-related changes by the Zbtb46 expression status, the hematopoietic system was investigated. Like the vascular system, the hematopoietic system in ZKO mice was mostly similar to the WT mice in homeostatic conditions when we investigated both the progenitor and committed cell types in lymphoid organs and peripheral blood, except with a slight increase in the Gr1+ cells in the PB (FIG. 9B). However, upon tumor challenge, the hematopoietic system in ZKO mice acquired more pronounced myeloid-biased pro-tumor immune characteristics as evidenced by the increased granulocyte-monocyte progenitor (GMP) and reduced pre-cDCs in both BM and spleen, as well as decreased T cells and cDCs and increased Gr1+ cells in the systemic circulation (FIG. 3B). In contrast, intra-tumor lentiviral Zbtb46 delivery, which restricted tumor growth and angiogenesis, led to a more anti-tumor immune microenvironment, reflected by the increased CTL/Treg and M1/M2 ratios, enhanced cDC1 and NK cell population, and reduced fibroblast activation in the TME (FIG. 3C).

To further understand the impact of Zbtb46 on the immune outcome, BM cells were isolated and cultured with a mixture of factors supporting DC and myeloid lineage development. Zbtb46 deficient BM cells generated reduced MHCII+DCs and increased Gr1+ myeloid cells compared to the WT BM cells (FIG. 3D). ZKO KSL (cKit+Sca1+Lin−) cell population that contains hematopoietic stem and progenitor cells (HSPC) produced more Gr1+ cells and fewer MHCII+DCs compared to the WT KSL cells, suggesting that the myeloid lineage skewing occurs at a more primitive progenitor level (FIG. 3E). Conversely, enforced Zbtb46 expression in BM cells reversed this trend to generate more DCs and fewer myeloid cells (FIG. 3D). Enforced Zbtb46 expression in BM-derived progenitors, mostly containing monocytes and lineage-negative cells, led to the generation of more DCs and fewer macrophages when transferred intra-tumor into an established tumor, a system that closely reflects the tumor-infiltrating monocyte differentiation (FIG. 3F). These findings suggested that Zbtb46 expression can control DC vs. myeloid differentiation output.

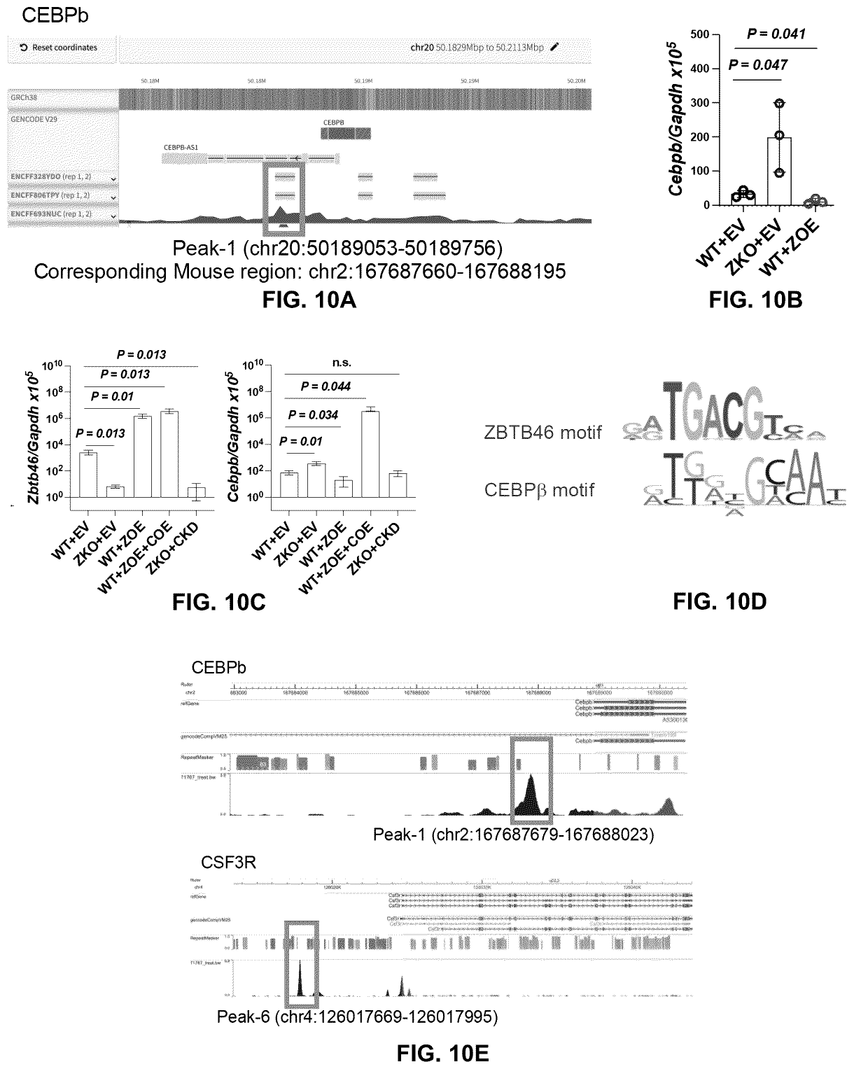

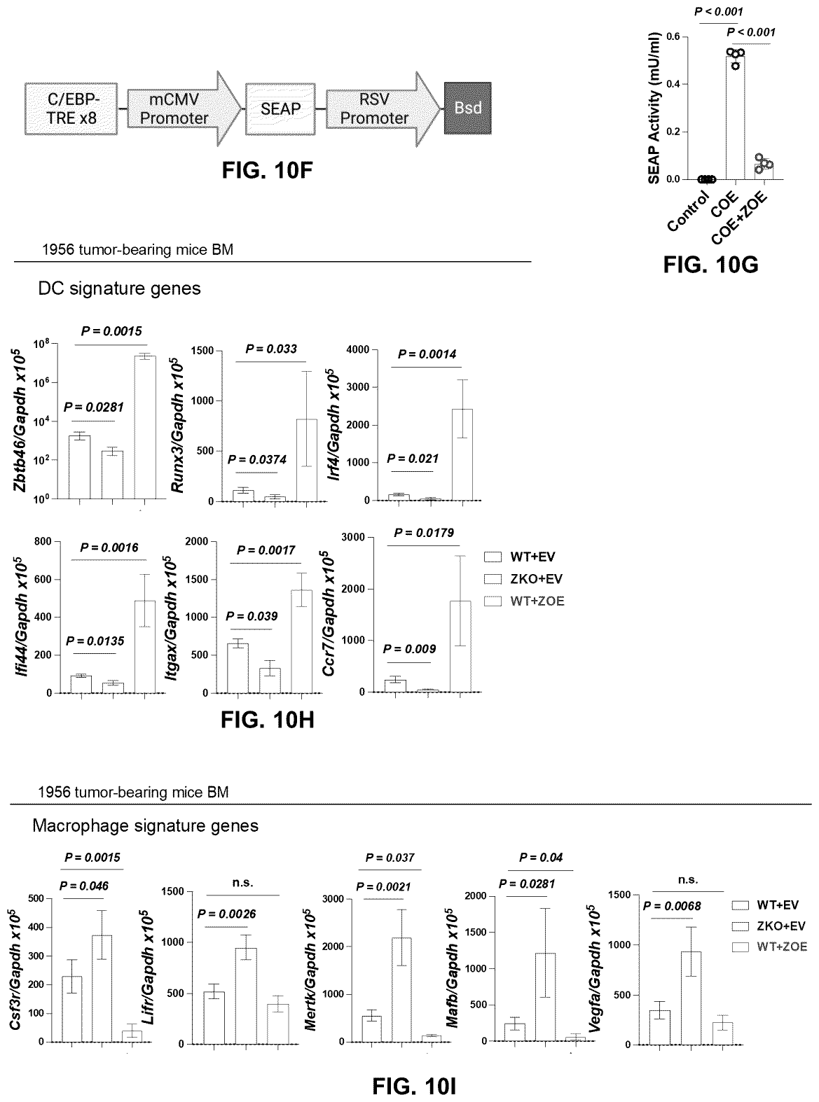

Examination of ZBTB46 ChIP-Seq and microarray data identified critical myeloid genes, including Cebpb, a crucial transcription factor for emergency granulopoiesis and monocyte/macrophage gene regulation, as potential direct targets of ZBTB46. By utilizing ChIP-qPCR, we confirmed that ZBTB46 could bind the Cebpb promoter region in BM-derived progenitors (FIG. 3G, FIG. 10A). While Cebpb expression was upregulated in the ZKO BM cells, enforced Zbtb46 expression downregulated Cebpb (FIG. 10B). Overexpression or knockdown of Cebpb partially reversed the overexpression or knockdown effect of Zbtb46, respectively, on myeloid and DC lineage generation from BM cells (FIG. 3H, FIG. 10C). These observations and the fact that the consensus DNA recognition site for both ZBTB46 and CEBPB is highly similar (FIG. 10D) raise the possibility that Cebpb can further upregulate myeloid genes that are targets of ZBTB46 repression in the absence of Zbtb46. Indeed, in the same cell system from ZKO mice, CEBPB binding to its' transcriptional targets, such as Cebpb itself and Csf3r, is significantly enhanced compared to the cells from WT mice (FIG. 3I, FIG. 10E).

A reporter system consisting of Cebpb transcriptional response element (TRE) further demonstrated that Zbtb46 can reduce the CEBPB-induced activation of TRE (FIG. 10F, FIG. 10G). This interplay between Zbtb46 and Cebpb is functionally important as BM cells from tumor-challenged ZKO mice had higher macrophage gene expression and lower DC gene expression than the WT tumor-challenged mice; enforced Zbtb46 expression reversed this expression pattern (FIG. 10H, FIG. 10I). These data suggest that the Zbtb46 regulation of Cebpb is at least partly responsible for the enforced Zbtb46-mediated immunostimulatory TME. Similar to the EC-specific overexpression outcome, intra-tumor transfer of Zbtb46-overexpressed BM-derived progenitors had partially restricted tumor growth (FIG. 3J), strengthening the notion of the collaborative nature of the cell-type-specific Zbtb46 expression in the tumor.

### Example 4-Therapeutic Maintenance of Zbtb46 Improves Cancer Immunotherapy Outcome

Background: To investigate the effect of Zbtb46 on cancer immunotherapy outcomes, the following experiments were conducted. Enforced Zbtb46 expression in the TME leads to vascular normalization and more DC generation with enhanced anti-tumor immunity. Because an immune-hot TME is a prerequisite for effective ICB therapy, we assessed whether the Zbtb46-maintenance could synergize with anti-PD1 immunotherapy.

Methods: The Zbtb46 mRNA-p5RHH peptide nanoparticle was prepared by using the Zbtb46 ORF sequence

(SEQ ID NO: 33)
ATGAACAACCGAAAGGAAGATATGGAAATCACTTCTCACTACCGGCATCT
GCTTCGAGAGCTCAATGAGCAGAGGCAGCACGGAGTCCTCTGTGATGCGT
GCGTCGTGGTGGAGGGCAAGGTCTTCAAGGCACATAAGAACGTCTTGCTT
GGGAGCAGCCGCTACTTTAAGACGCTCTACTGCCAGGTACAGAAGACATC
TGACCAGGCCACCGTCACTCACTTGGACATTGTTACAGCCCAGGGCTTCA
AGGCCATTATTGACTTCATGTACTCCGCCCATCTGGCTCTCACTAGTAGG
AATGTCATCGAGGTGATGTCAGCTGCCAGCTTCCTACAGATGACTGACAT
TGTGCAGGCCTGCCATGATTTCATCAAGGCTGCACTGGACATCAGCATAA
AGTCAGATGCCTCCGATGAACTCTCAGAATTTGAGATTGGCACCCCAGCC
AGCAACAGTACAGAGGCGTTGATCTCAGCTGTGATGGCTGGAAGGAGTAT
CTCCCCATGGTTGGCTCGGAGAACAAGTCCTGCCAATTCTTCTGGAGACT
CTGCCATTGCCAGCTGTCATGAAGGAGGAAGCAGCTATGGGAAGGAGGAC
CAGGAACCCAAAGCTGATGGCCCTGATGACGTTTCTTCACAGTCTTTGTG
GCCTGGAGATGTAGGCTATGGGTCTCTGCGCATCAAGGAAGAACAGATTT
CACCATCACATTATGGAGGGAGTGAGCTTCCATCTTCCAAGGACACTGCA
ATACAGAATTCTTTATCAGAACAGGGTTCTGGGGATGGCTGGCAGCCCAC
AGGCCGGAGGAAGAATCGGAAAAACAAAGAGACTGTCCGACACATCACCC
AGCAGGTGGAGGAGGACAGCCAGGCTGGCTCTCCAGTACCTTCATTCCTA
CCCACATCGGGATGGCCTTTCAGCAGCCGAGACTCAAATGTAGACCTGAC
GGTCACTGAGGCCAGCAGCTTGGACAGCCGAGGCGAGAGAGCAGAGCTCT
ATGCTCACATCGATGAGGGCCTACTAGGAGGAGAAACCAGCTACTTGGGC
CCACCCCTCACCCCAGAGAAGGAAGAAGCACTACACCAGGCTACTGCAGT
GGCCAATCTTCGTGCTGCACTCATGAGTAAGAACAGTCTGCTGTCACTCA
AGGCTGACGTGCTCGGTGATGATGGCTCACTTCTGTTCGAGTACCTGCCC
AAAGGTGCCCACTCACTGTCTCGTAAGTGCAAGTTCTGGTGTGTCACTGT
GTCTTCCTTTGGTTTAAGCACCTCAGTTCAGCCCTTCAGACCCTGGAGTC
ACTGA,

which was made into a modified mRNA transcript with complete substitution of pseudo-U in RNase-free water from TriLink Biotechnologies. CleanCap® EGFP mRNA (Cat: L-7201-100) was used as mRNA control. 8 ml (1 mg/ml) of the mRNA solution was mixed with 5 ml of 20 mM p5RHH peptide solution and 187 ml of 1×HBSS (Gibco) to prepare the nanoparticle complex and immediately injected into the mouse through the tail vein.

Tumor transplantation studies were conducted by mixing 1 ml of growth factor reduced Matrigel (Cat: 354248; Corning) with 1 ml of cultured LLC-GFP tumor cell suspension (2×10⁶/ml in PBS); 100 ml of the mixture was subcutaneously injected into the back of the mice. 1956- and 1969-sarcoma cells were subcutaneously injected as 1×10⁶ cells in 150 ml PBS+Matrigel solution (1:1) per mouse to the flank of the mice. PyMT-BO1 cells were orthotopically injected as 1×10⁵ cells in 50 ml PBS+Matrigel solution (1:1) per mouse to the mammary fat pad of the mice. Palpable tumors started to develop 4-5 days after transplantation and tumor growth was measured until the end of the study. For overexpression studies, relevant lentiviral particles were intra-tumor injected as 15 ml/injection for a viral content of 2×10\hat{ }6 TU/injection, as many times as indicated in the relevant figures. For in vivo treatment studies, rat IgG2ak anti-mouse PD1 antibody (Cat: P372, Clone: RMP1-14, Leinco Technologies) was injected intraperitoneally at a dose of 200 mg/day, and DC101 (anti-VEGFR2) was injected intraperitoneally at a dose of 40 mg/kg (Cat: BE0060, Bio X cell) (76). Rat IgG2a isotype control at an equivalent dose was used as control.

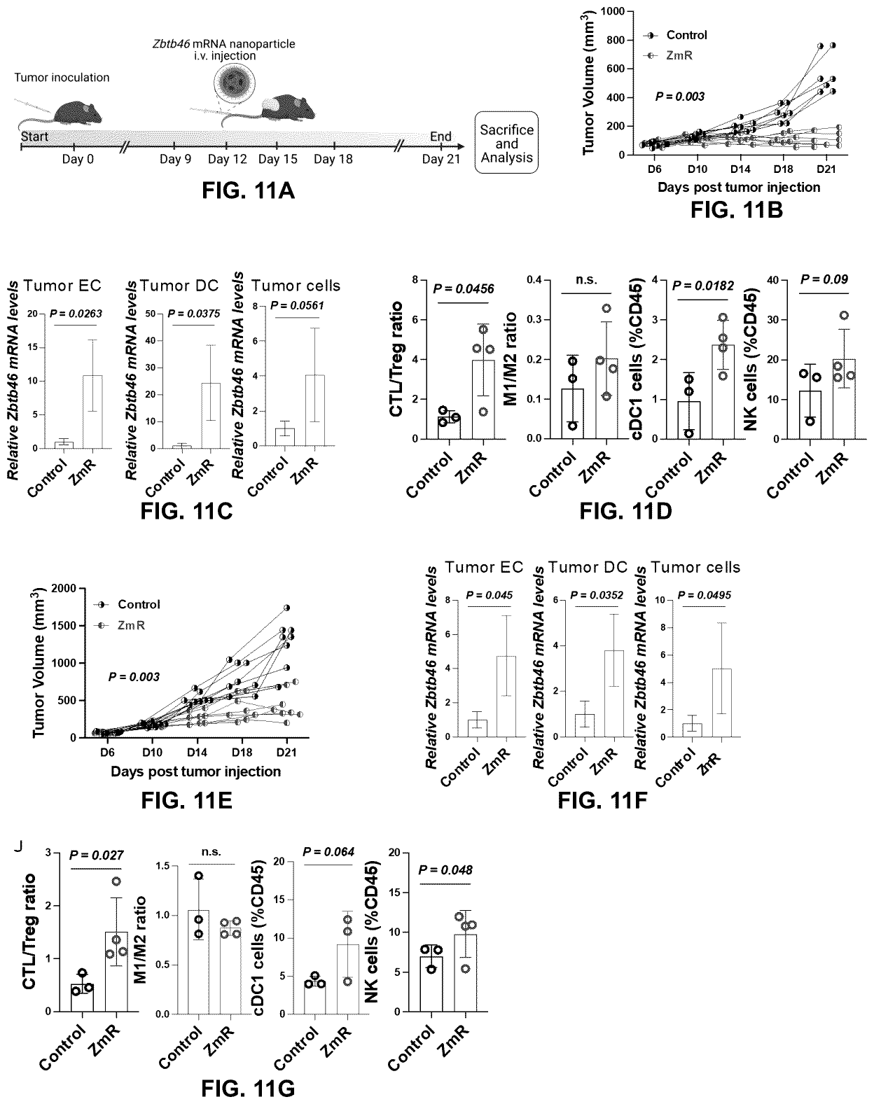

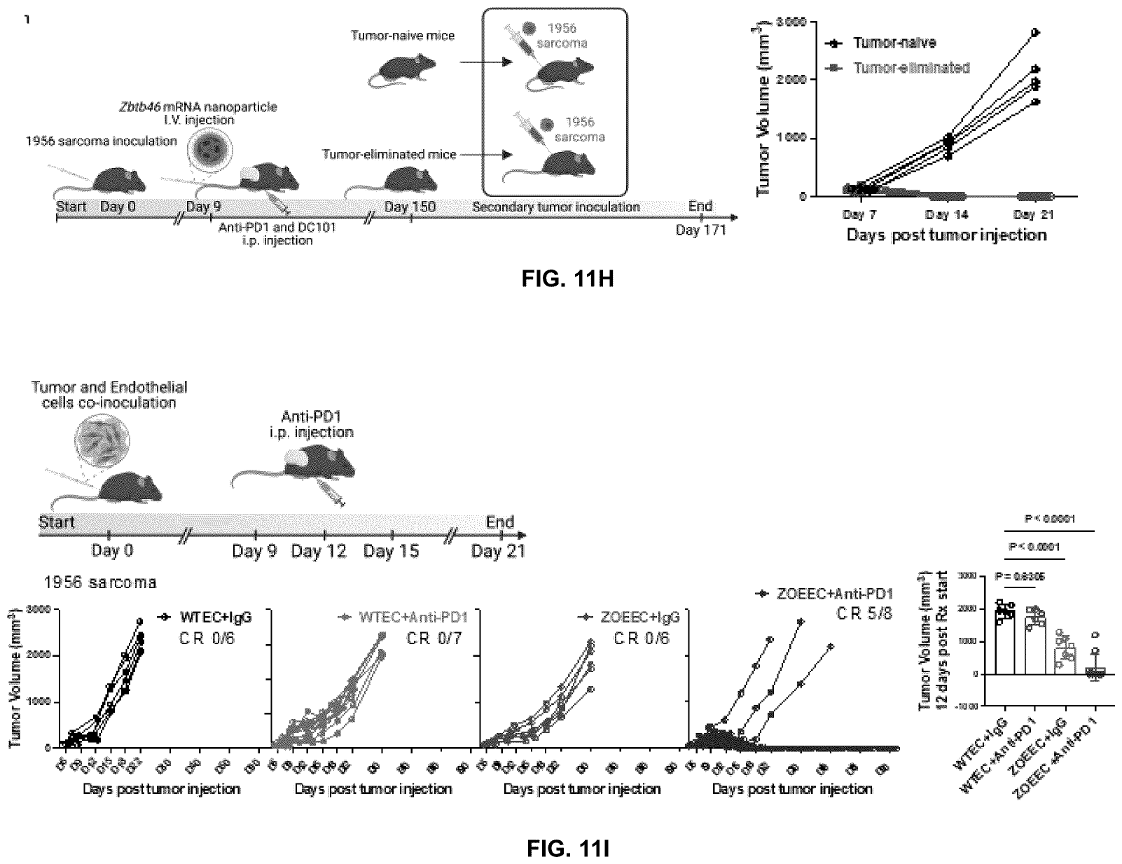

Results: A Zbtb46 mRNA nanoparticle was generated using the p5RHH peptide system. The p5RHH peptide-based nanoparticle system readily formulates mRNA for systemic administration and extrahepatic nucleotide delivery and showed promise for safe and effective clinical translation. Systemic administration of Zbtb46 mRNA nanoparticle was effective in sustaining Zbtb46 expression in tumor-DCs and -ECs and resulted in the restriction of tumor growth (FIG. 11A, FIG. 11B, FIG. 11C, FIG. 11D, FIG. 11E, FIG. 11F, FIG. 11G). Although tumor cells also acquired Zbtb46 expression, tumor cells overexpressing Zbtb46 do not show much growth differences compared to parental lines (FIG. 7D), suggesting the EC- and DC-focused Zbtb46 delivery as the driver for the observed tumor control.

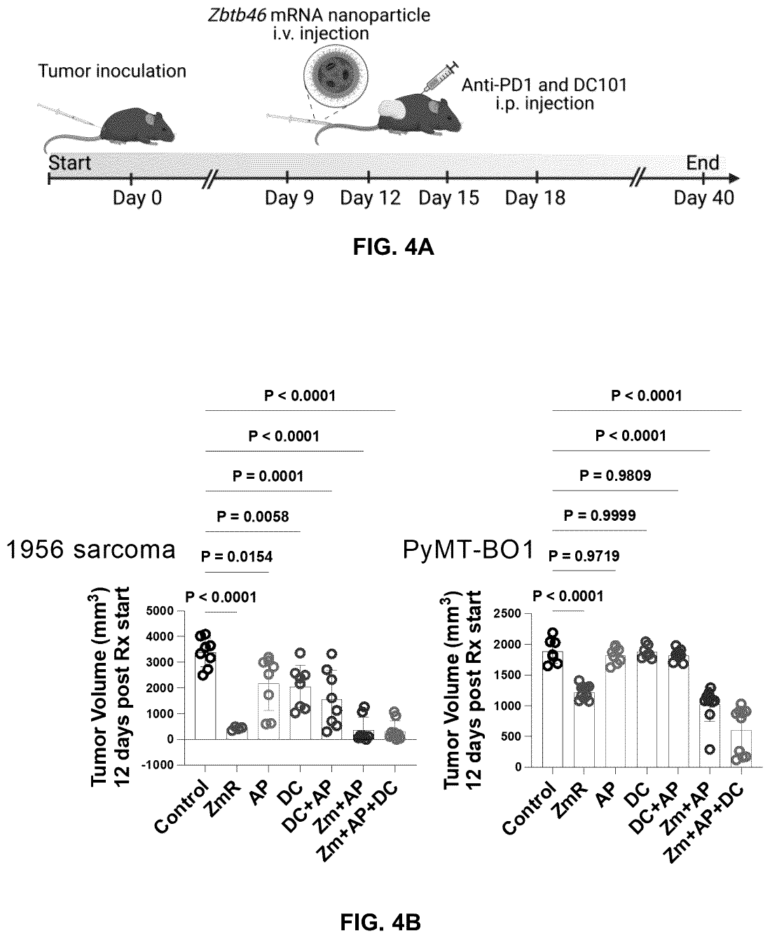

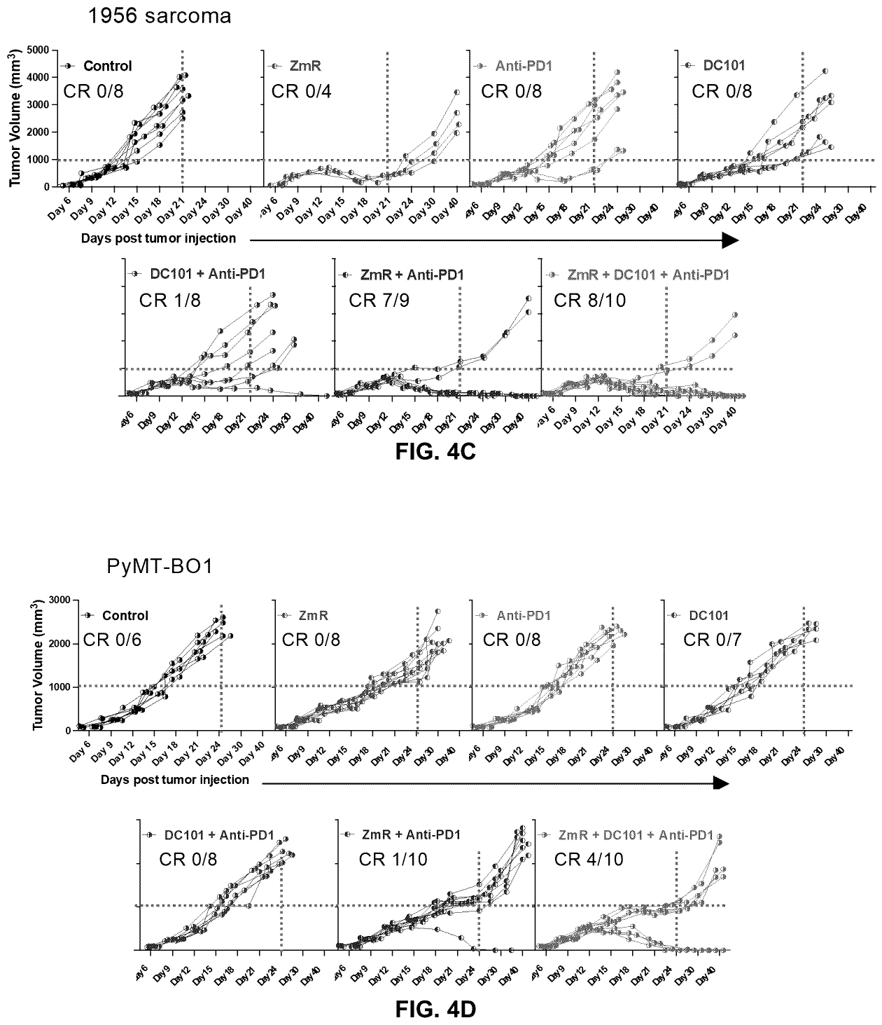

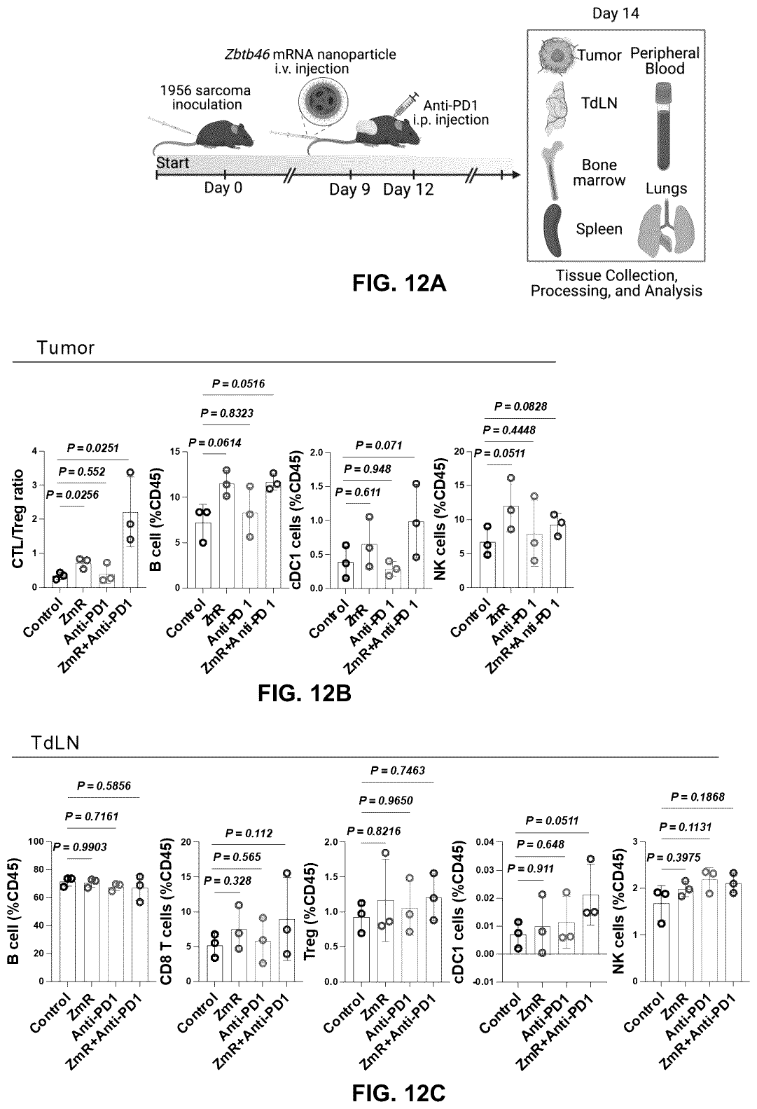

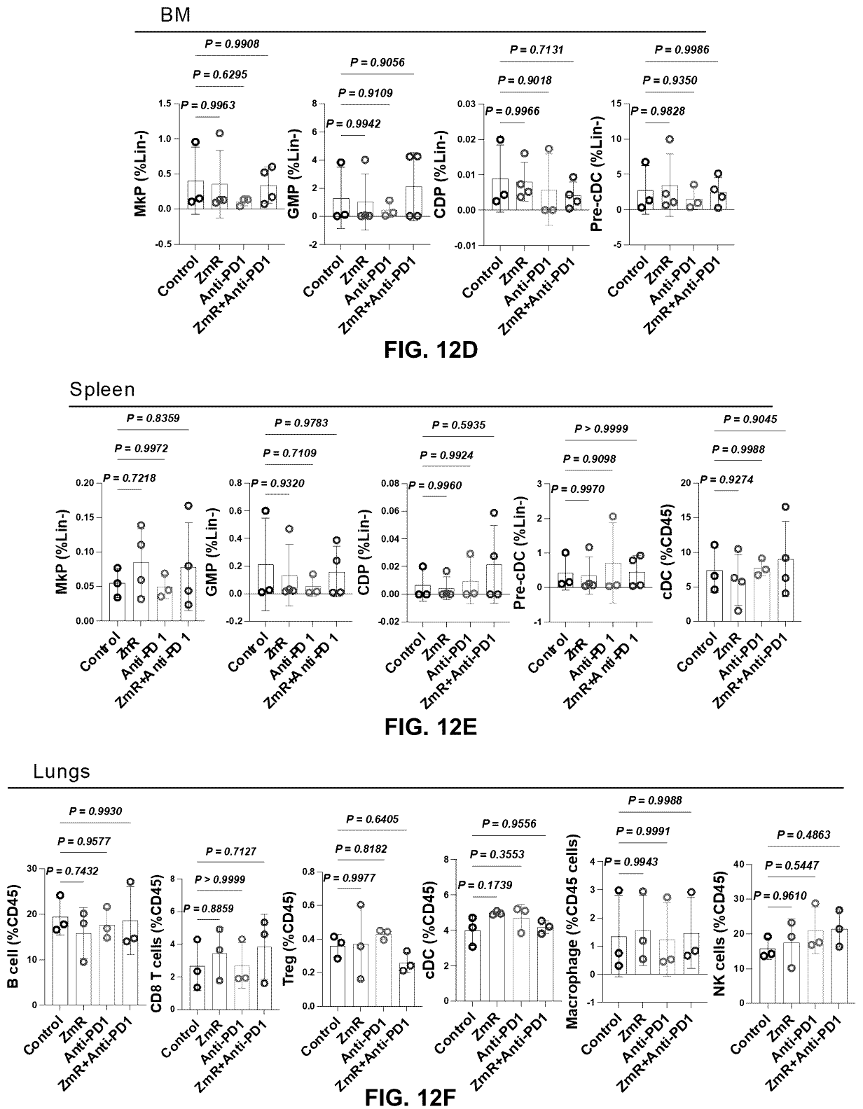

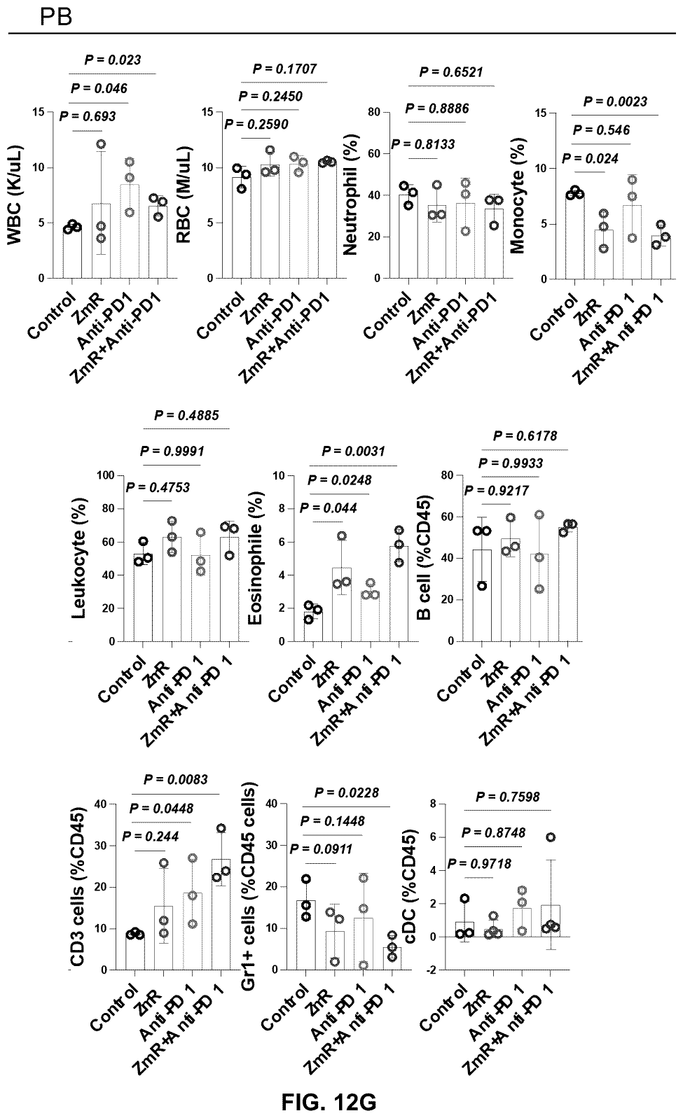

The tumor growth restriction was associated with an immunostimulatory TME (FIG. 11A, FIG. 11B, FIG. 11C, FIG. 11D, FIG. 11E, FIG. 11F, FIG. 11G). Remarkably, the nanoparticles induced dramatic response in both anti-PD1-responsive (1956 sarcoma) and -refractory (PyMT-BO1) tumor models following the treatment, generating long-term complete remission of tumor mass in many of the treated animals challenged with the anti-PD1-responsive 1956 sarcoma tumor. The addition of a VEGFR2 inhibitor (DC101) enhanced the treatment response hinting at the collaborative nature of the targeted Zbtb46 and Vegf pathways in the tumor (FIG. 4A, FIG. 4B, FIG. 4C, FIG. 4D). The tumor-eliminated mice spontaneously rejected the secondary challenge with the same tumor, indicative of the development of long-term immunological memory (FIG. 11H). Intriguingly, co-transplanting tumor cells with Zbtb46-overexpressed ECs also improved the anti-PD1 treatment outcome (FIG. 11I), reinforcing the importance of the EC-specific Zbtb46 expression in tumor progression. Post-treatment analysis of the TME, lymphoid organs, peripheral blood, and a distal non-tumor organ revealed that the immunostimulatory impact of the systemic nanoparticle treatment was primarily restricted to the TME (FIG. 12A, FIG. 12B, FIG. 12C, FIG. 12D, FIG. 12E, FIG. 12F, FIG. 12G).

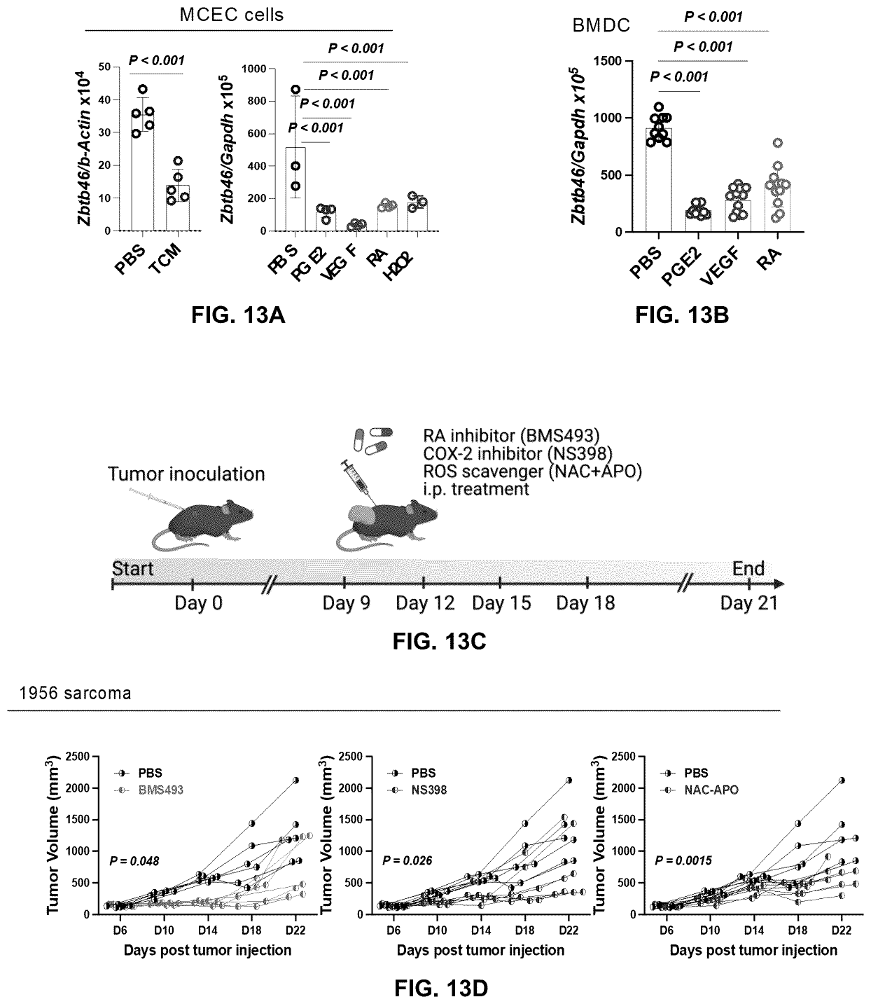

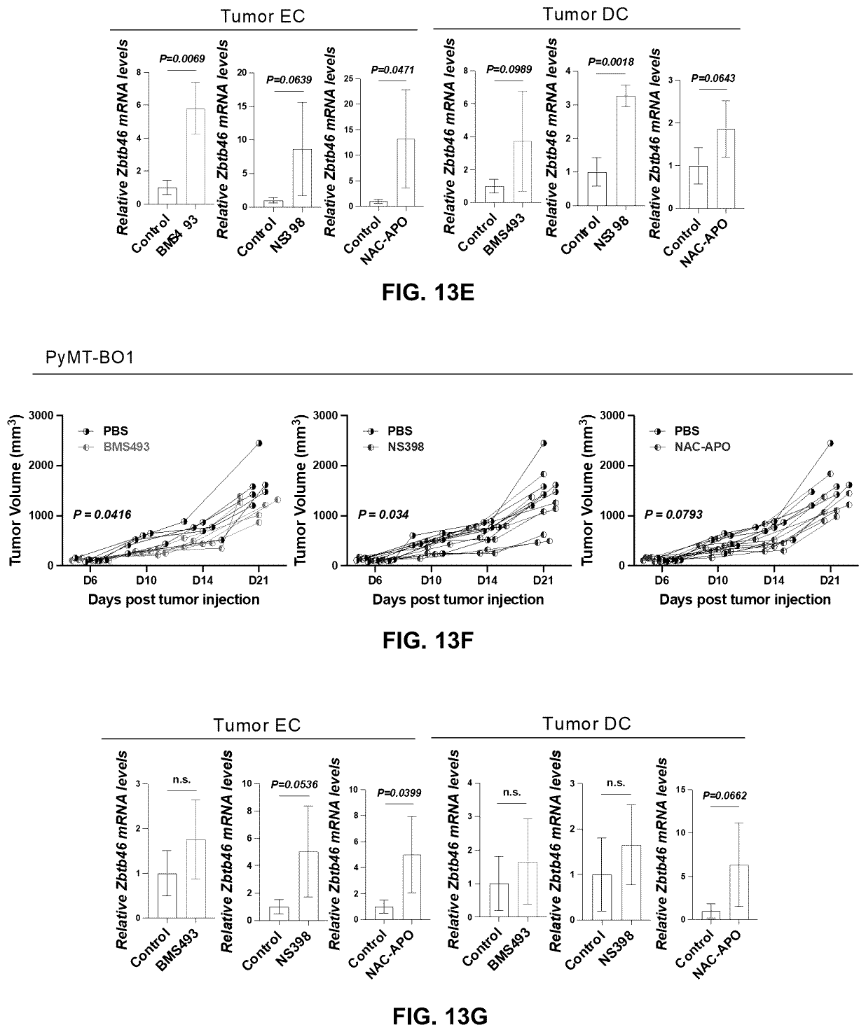

Finally, tumor-derived factors are likely to downregulate Zbtb46 in the TME. Indeed, tumor-conditioned media (TCM) can downregulate Zbtb46 expression in vitro (FIG. 13A). Among the factors produced by the tumor, the reactive oxygen species (ROS), prostaglandin E2 (PGE2), retinoic acid (RA), and vascular endothelial growth factors (VEGF) downregulated Zbtb46 expression in both EC and DC (FIG. 13A, FIG. 13B). Pharmacological targeting of the PGE2, RA, and ROS pathways using FDA-approved and commercially available inhibitors modestly prevented tumor-mediated Zbtb46 suppression both in EC and DC and partially restricted tumor growth (FIG. 13C, FIG. 13D, FIG. 13E, FIG. 13F, FIG. 13G), further hinting at the translatability of regulating Zbtb46 in the tumor.

What is claimed is:

1. A method to treat a cancer in a patient, the method comprising administering a therapeutically effective amount of an immunotherapy and a therapeutically effective amount of a Zbtb46 modulation agent(s) to the patient.

2. The method of claim 1, wherein the immunotherapy comprises an immune checkpoint blockade (ICB) therapy.

3. The method of claim 1, wherein the ICB therapy comprises an anti-PD1 therapy.

4. The method of claim 1, wherein the Zbtb46 modulation agent increases expression of Zbtb46 in a cancer patient's tumor microenvironment, comprising a Zbtb46 overexpression construct.

5. The method of claim 1, wherein the Zbtb46 modulation agent normalizes tumor blood vessels.

6. The method of claim 1, wherein the Zbtb46 modulation agent comprises a Cebpb antibody, wherein the Cebpb antibody suppresses Cebpb transcription.

7. The method of claim 1, wherein the Zbtb46 modulating agent comprises any one of BMS493, NS398, NAC+APO, or DC101, wherein the Zbtb46 modulating agent suppresses tumor-derived factors.

8. The method of claim 1, wherein the Zbtb46 modulation agent comprises a Cebpb shRNA construct or Cebpb antibody, wherein the Zbtb46 modulation agent enhances the generation of dendritic cells relative to macrophages from bone-marrow precursors.

9. The method of claim 1, wherein the Zbtb46 modulation agent enhances an immune response in a cancer patient's tumor.

10. The method of claim 1, wherein the Zbtb46 modulation agent comprises a Zbtb46 mRNA.

11. The method of claim 1, wherein the Zbtb46 modulation agent comprises a Zbtb46 mRNA nanoparticle.

12. A method of selecting a treatment for a cancer patient in need, the method comprising
a. performing RNAseq on a biopsy of a tumor of the patient;
b. quantifying an expression of Zbtb46 in the patient's tumor; and
c. selecting a treatment of an immunotherapy and an adjuvant therapy for the patient if the expression of Zbtb46 falls below a standard threshold.

13. The method of claim 12, wherein the adjuvant therapy comprises a Zbtb46 modulation agent.

14. The method of claim 12, wherein the Zbtb46 modulation agent comprises a Zbtb46 mRNA.

15. The method of claim 12, wherein the immunotherapy comprises an ICB therapy.

16. The method of claim 12, wherein the ICB therapy comprises an anti-PD1 therapy.

17. The method of claim 12, further comprising identifying the standard threshold by comparing a Zbtb46 expression in patients that respond to the immunotherapy to a corresponding Zbtb46 expression in patients that do not respond to immunotherapy, and selecting the threshold expression value that most differentiates the patients that do not respond to the immunotherapy from the patients that do respond to the immunotherapy.
